(19) United States
(12) Patent Application Publication
(10) Pub. No.: US 2024/0238616 A1
(43) Pub. Date: Jul. 18, 2024

(54) RADIOSENSITIZING COMPOSITIONS AND METHODS

(71) Applicant: THE JACKSON LABORATORY, Bar Harbor, ME (US)

(72) Inventor: Roel VERHAAK, Farmington, CT (US)

(21) Appl. No.: 18/563,207

(22) PCT Filed: May 26, 2022
(86) PCT No.: PCT/US2022/031111

§ 371 Date: Nov. 21, 2023

Related U.S. Application Data
(60) Provisional application No. 63/193,432, filed on May 26, 2021.

(51) Int. Cl. A61N 5/10

(52) U.S. Cl. CPC A61N 5/1001; A61N 5/1077; A61N 2005/1024; A61N 2005/1098

(57) ABSTRACT

The invention relates, in part, to methods and compounds for radiosensitizing cells and increasing efficacy of treatments for cancers.

### RELATED APPLICATIONS

This application claims benefit under 35 U.S.C. § 119(e) of U.S. Provisional application Ser. No. 63/193,432, filed May 26, 2021, the contents of which is incorporated by referenced herein in its entirety.

### GOVERNMENT SUPPORT

This invention was made with government support under NIH/NCI R01 CA190121, R01 CA237208, NIH/NINDS R21 NS114873 awarded by the National Institutes of Health and W81XWH1910246 awarded by the Department of Defense. The government has certain rights in the invention.

### FIELD OF THE INVENTION

The invention relates, in part, to methods and compounds for radiosensitizing cells and increasing efficacy of treatments for cancers.

### BACKGROUND OF THE INVENTION

Radiation therapy or radiotherapy is a main stay of cancer therapy, used in clinical management of more than half of cancer patients. In certain subjects, therapy-resistant tumors are present. There are currently few ways to enhance efficacy of radiation therapies, which limits effectiveness of some cancer therapeutics.

### SUMMARY OF THE INVENTION

According to an aspect of the invention, a method of radio-sensitizing a cell is provided, the method including contacting the cell with an exogenous cytidine deaminase inhibitor compound. In some embodiments, the exogenous cytidine deaminase inhibitor compound is an inhibitor compound capable of inhibiting activity of an apolipoprotein B mRNA editing enzyme, catalytic polypeptide-like (APOBEC) enzyme. In some embodiments, the APOBEC enzyme is one or more of: an APOBEC1 enzyme, an APOBEC3A enzyme, an APOBEC3B enzyme, an APOBEC3C enzyme, an APOBEC3D enzyme, an APOBEC3E enzyme, an APOBEC3F enzyme, an APOBEC3G enzyme, an APOBEC3H enzyme, and an activation-induced cytidine deaminase (AID) enzyme. In some embodiments, the cell is a cancer cell. In some embodiments, the method also includes contacting the cell with radiation. In some embodiments, the method also includes contacting the cell with two or more exogenous cytidine deaminase inhibitor compounds. In some embodiments, the cell is in a subject. In some embodiments, the contacting includes administering the exogenous cytidine deaminase inhibitor compound to the subject. In some embodiments, the subject has a cancer. In some embodiments, the cancer is a cancer with elevated APOBEC activity. In some embodiments, the cancer is a bone cancer, a soft tissue cancer, a colon cancer, a rectal cancer, an esophageal cancer, a lung cancer, a central nervous system (CNS) cancer, or uterine cancer. In some embodiments, the cancer is breast cancer. In some embodiments, the method also includes administering a radiotherapy to the subject. In some embodiments, the radiotherapy comprises external beam radiation. In some embodiments, the radiotherapy includes brachytherapy. In some embodiments, the subject is a vertebrate, optionally a mammal. In some embodiments, the subject is a human. In some embodiments, the exogenous cytidine deaminase inhibitor compound is in a pharmaceutical composition. In some embodiments, the pharmaceutical composition also includes a pharmaceutically acceptable carrier.

According to another aspect of the invention, a method of enhancing efficacy of a radiotherapy administration in a subject is provided, the method including: administering to a subject in need of such treatment, an effective amount of a exogenous cytidine deaminase inhibitor compound, wherein (i) the subject is administered a radiotherapy; (ii) the exogenous cytidine deaminase inhibitor compound is administered in a therapeutic regimen; and (iii) the administered exogenous cytidine deaminase inhibitor compound enhances the efficacy of the administered radiotherapy in the subject. In some embodiments, the exogenous cytidine deaminase inhibitor compound is capable of inhibiting an activity of an apolipoprotein B mRNA editing enzyme, catalytic polypeptide-like (APOBEC) enzyme. In some embodiments, the APOBEC enzyme is one or more of: an APOBEC1 enzyme, an APOBEC3A enzyme, an APOBEC3B enzyme, an APOBEC3C enzyme, an APOBEC3D enzyme, an APOBEC3E enzyme, an APOBEC3F enzyme, an APOBEC3G enzyme, an APOBEC3H enzyme, and an activation-induced cytidine deaminase (AID) enzyme. In some embodiments, the therapeutic regimen comprises administering the exogenous cytidine deaminase inhibitor compound prior to or concurrent with the administered radiotherapy. In some embodiments, the therapeutic regimen includes administering the exogenous cytidine deaminase inhibitor compound to the subject prior to and concurrently the administered radiotherapy. In some embodiments, the subject is a vertebrate, optionally a mammal. In some embodiments, the subject is a human. In some embodiments, the enhancing increases a level of cell death in the subject compared to a control level of cell death. In some embodiments, the cell death comprises cancer cell death. In some embodiments, the control level of cell death is a level of cell death in a subject or plurality of subjects administered the radiotherapy and not administered the exogenous cytidine deaminase inhibitor compound therapeutic regimen. In some embodiments, the radiotherapy includes external beam radiation. In some embodiments, the radiotherapy includes brachytherapy. In some embodiments, the subject has a cancer. In some embodiments, the cancer is a cancer with elevated APOBEC activity. In some embodiments, the cancer is a bone cancer, a soft tissue cancer, a colon cancer, a rectal cancer, an esophageal cancer, a lung cancer, a central nervous system (CNS) cancer, or uterine cancer. In some embodiments, the cancer is breast cancer. In some embodiments, the exogenous cytidine deaminase inhibitor is administered to the subject in a pharmaceutical composition. In some embodiments, the pharmaceutical composition also includes a pharmaceutically acceptable carrier.

### BRIEF DESCRIPTION OF THE DRAWINGS

FIG. 1A-D provides graphs illustrating that radiotherapy was associated with an increased small deletion burden. FIG. 1A shows a boxplot depicting the burden of newly acquired/post-treatment small deletions (deletions/Mb) in RT-naïve (RT−, n=34) and RT-received (RT+, n=156) patients from the GLASS cohort. Mann-Whitney U test was applied for statistical testing. FIG. 1B shows violin plots of longitudinal comparison of small deletion burden between primary and recurrent glioma samples, separated by hypermutation (HM) and Radiotherapy (RT). Paired Wilcoxon signed-rank test was applied for statistical testing. FIG. 1C shows a forest plot showing a multivariable log-linear regression model of newly acquired small deletion burden (deletions/Mb) including (1) temozolomide (TMZ)-treatment, (2) hypermutation (HM), (3) RT-treatment, (4) molecular subtype and (5) surgical interval (in months) as variables. OR, odds ratio; CI, confidence interval. FIG. 1D, upper row, shows boxplots depicting small deletion burden (deletions/Mb) in in metastatic cohort tumor samples separated by primary tumor location. For each individual panel of three boxplots, RT-naïve (RT−, left), RT-treated with palliative intent (RT+ pal, middle), and RT-treated with curative intent (RT+ cur, right). Statistical testing, Kruskal-Wallis test. FIG. 1D, lower row, shows bar graphs depicting sample sizes of the metastatic cohort separated by primary tumor location. Each bar graph panel corresponds to the boxplot panel immediately above.

FIG. 2A-J provides graphs and a flowchart illustrating the association of radiotherapy with an increased burden of newly acquired/post-treatment mutations. FIG. 2A shows boxplots comparing the burden of several types of newly acquired/post-treatment mutations (mutations/Mb) in RT-naïve (RT−, n=34) and RT-treated (RT+, n=156) patients from the GLASS cohort. Mutations were separated by small deletions (DEL), small insertions (INS), and single nucleotide variants (SNV). Statistical testing, Mann-Whitney U test. FIG. 2B shows boxplots comparing newly acquired small deletion burdens (mutations/Mb) between RT-naïve (RT−) and RT-treated (RT+) cases separated by molecular subtype (IDHmut vs. IDHwt). Statistical testing, Mann-Whitney U test. FIG. 2C shows boxplots comparing the mean cancer cell fractions of small deletions per patient in the GLASS cohort, separated by P (primary-only fraction, pretreatment), S (shared fraction, pre-treatment), and R (recurrence only fraction, post-treatment), and by HM (hypermutation) versus non-HM (non-hypermutation) status. Statistical testing, Mann-Whitney U test. FIG. 2D shows forest plots showing a multivariable log-linear regression model of newly acquired mutation burdens (mutations/mb) in the GLASS cohort for the following variables: TMZ-treatment, hypermutation, RT-treatment, molecular subtype, and surgical interval (in months). Mutation types were separated into small deletions (circle), small insertions (square), indels (diamond, small deletions+small insertions), SNVs (triangle, single nucleotide variants), and overall tumor mutational burden (inverted triangle; TMB, small indels+SNVs). A point indicates a mean estimate of the model; lines indicate 95% confidence intervals. Hypermutation was significantly associated with increased burden of all types of mutations, and RT was associated with a slightly increased burden of small deletions and indels (potentially driven by the large effect size of small deletions). FIG. 2E shows a flowchart of sample selection and filtering criteria for the metastatic cohort. FIG. 2F shows boxplots comparing small deletion burdens between RT−, RT+ pal, and RT+ cur samples, respectively, for breast, lung, and bone/soft tissue cancers separated into their respective subtypes. Statistical testing, Kruskal-Wallis test. FIG. 2G shows boxplots depicting small deletion burdens in HRD−/MSI− (n=3,413), HRD+ (n=218), and MSI+ (n=62) samples from the HMF cohort separated by radiotherapy treatment status (homologous recombination deficiency (HRD), microsatellite instability (MSI)). Statistical testing, Mann-Whitney U test. FIG. 2H shows forest plots depicting a multivariable log-linear regression model for mutation burdens in the metastatic cohort. Mutations were separated into small deletions, small insertions, and SNVs. Independent variables included age, tumor type (primary tumor location), DNA repair deficiency background, and various treatment types including radiotherapy, taxane, alkylating agents, platin, and others. FIG. 2I shows boxplots comparing small deletion counts between control vs. ionizing radiation groups from a previously described dataset [Kucab et al., Cell 177, 821-836.e16. (2019)]. Statistical testing, Mann-Whitney U test. FIG. 2J shows a bar graph of the distribution of small deletion counts per treatment group from a previously described dataset [Kucab et al., Cell 177, 821-836.c16. (2019)]. Bars indicate means, error bars reflect standard deviation, and dots indicate the median count of small deletions. The ionizing radiation group displayed the highest median counts of small deletions. PAH, polycyclic aromatic hydrocarbon; ROS, reactive oxygen species; UV, ultraviolet; DDR, DNA damage response.

FIG. 3A-D presents graphs and a schematic illustrating distribution of small deletion characteristics. FIG. 3A shows graphs illustrating the length distribution of small deletion characteristics in the GLASS cohort. The upper panel graphs compare mean deletion lengths in primary vs. recurrent IDH mutant glioma (n=81), separated by RT-treatment (RT−, n=49; RT+, n=32). Statistical testing, paired Wilcoxon signed-rank test. A significant increase in mean deletion lengths was only observed for ionizing radiation treated samples. The lower panel graph shows deletion proportions calculated for each patient, comparing Primary and Recurrence in non-hypermutant glioma treated with RT (n=44). Y-Axis, proportion of deletions; X-Axis, deletion length >1 bp; mean (point) and 95% CI (line-range). Statistical testing, paired Wilcoxon signed-rank test (*=p<0.05, **=p<0.01). FIG. 3B shows graphs illustrating the deletion length distribution in the metastatic cohort. The boxplots of the upper panel graph compare mean deletion lengths in RT-naïve (RT−), RT+ pal, and RT-cur samples. Kruskal-Wallis test was applied for statistical testing. The lower panel graph shows deletion proportions calculated for each patient, comparing RT-naïve (RT−), RT+ pal, and RT-cur samples. Y-Axis, proportion of deletions; X-Axis, deletion length >1 bp; mean (point) and 95%-CI (line-range). Statistical testing, Kruskal-Wallis test (*=p<0.05, **=p<0.01, ***=p<0.001, ****=p<0.0001). In lower panel: RT−, diamond; RT+ pal, dot with black border and light gray fill; RT+ cur, dark dot. FIG. 3C shows graphs depicting relations to genomic features in the GLASS cohort. The upper panel forest plot shows distributions of deletions in relation to genomic features. Y-Axis, non-B-DNA genomic feature; X-Axis, log 10 ratio of mean distance of non-radiation-associated and radiation-associated post-treatment deletions to genomic feature over background distribution in non-hypermutated glioma samples (n=69). Distribution of radiation-associated deletions showed little variability (narrow 95% CI) and resembled background distribution more closely (closer to 0). Significant differences between radiation-associated deletions and non-radiation-associated deletions were seen in relation to repeats (Mann-Whitney U test). In upper panel: RT−, light gray dot with asterisk; RT+, dark dot. The lower panel line graph shows the empirical cumulative distribution function (ECDF, Y-Axis) of distance to non-B-DNA features in kb (X-Axis), revealing a right-shift towards larger distances in post-radiated non-hypermutated recurrent samples (n=44). Longitudinal differences were not observed in either hypermutated or RT-naïve non-hypermutated glioma samples (additional data shown in FIG. 4C). In lower panel: A-phased repeat, lines with filled circles; direct repeat, lines with open squares; G-quadruplex motif, lines with filled squares; inverted repeat mirror, lines with open circles; repeat, lines with X's; short tandem repeat, lines with open triangles; and Z-DNA motif, lines with asterisks. Solid lines (all instances), primary; dashed lines (all instances), recurrence. FIG. 3D shows a schematic and graph illustrating categorization of small deletions in the GLASS cohort. The schematic (upper panel) depicts separating small deletions in the GLASS cohort into three major categories: 1 bp, >1 bp without microhomology, and >1 bp with microhomology in IDH mutant gliomas (n=81). The microhomology category was further classified based on the occurrence of microhomology repeat sequences and length of repeats. The graph (lower panel) compares the proportion of deletions for each non-hypermutated glioma sample treated with RT (n=44, further comparisons shown in Supp. FIG. 2E) using the paired Wilcoxon signed-rank test. Deletions of 1 bp length significantly decreased, whereas deletions >1 bp without microhomology significantly increased in response to RT. In FIG. 2D: >1 bp no microhomology (MH), n=3,325; 1 bp, n=11, 303; and >1 bp with MH, n=4,325.

FIG. 4A-F provides graphs illustrating comparisons of deletion data in various samples. FIG. 4A compares mean deletion lengths of newly acquired deletions (post-treatment fraction) in RT− vs RT+ IDHmut glioma samples. Statistical testing, Mann-Whitney U test. FIG. 4B shows mean deletion lengths in RT-naïve (RT−), palliative RT-treated (RT+ pal), and curative RT-treated (RT+ cur) tumor samples separated by primary tumor location in the metastatic cohort. Statistical testing, Kruskal-Wallis test. FIG. 4C shows longitudinal comparisons of mean distances of deletions of non-B DNA features in kb (X-Axis) in IDHmut glioma cases (Y-Axis). Cases were separated by radiation treatment status and hypermutation status. Note that neither in hypermutated, nor in RT-naïve non-hypermutated glioma samples significant longitudinal differences were observed. Statistical testing, paired Wilcoxon signed-rank test. FIG. 4D shows gene-wise dN/dS estimates by radiation treatment (rows) and fraction (columns) in the GLASS cohort. Genes are sorted by Q value (Bonferroni adjusted P value) and P value; Q values are indicated with bars. A vertical line indicates the Q value threshold of 0.05. No genes showed significant selection in the post-radiation fraction. FIG. 4E compares the proportion of deletions for IDHmut glioma samples separated by radiation treatment and hypermutation using the paired Wilcoxon signed-rank test. For each sample, the proportion of deletions with 1 bp length, >1 bp length with microhomology, and >1 bp length without microhomology add up to 1. [The lower right three panels (RT+non-hypermutators) are reproduced from FIG. 2D for comparison with other groups.] 1 bp deletions were significantly increased in hypermutated radiation-naïve cases. No significant differences were observed for radiation-naïve non-hypermutated cases. FIG. 4F shows comparisons of deletion proportions in the metastatic cohort between RT-treated (RT+ pal and RT+ cur) and RT-naïve (RT−) cases using the Kruskal-Wallis test. In bone/soft tissue, breast, head and neck, and nervous system cancers, significantly lower proportions of deletions >1 bp with microhomology were observed in RT-treated samples compared to RT-naïve samples. In contrast, RT-treated breast, colon/rectum, esophagus, nervous system, and prostate tumor samples showed significantly higher proportions in deletions >1 bp without microhomology.

FIG. 5A-B provides graphs illustrating ID8 and APOBEC-SBS signatures associated with radiotherapy. FIG. 5A shows indel (ID) and single base substitution (SBS) mutational signatures in the GLASS cohort associated with RT (radiotherapy), hypermutation (HM), microsatellite instability (MSI), and homologous recombination deficiency (HRD). FIG. 5B shows indel (ID) and single base substitution (SBS) mutational signatures in the HMF cohort associated with RT (radiotherapy), hypermutation (HM), microsatellite instability (MSI) and homologous recombination deficiency (HRD). Statistical testing for both FIG. 5A and FIG. 5B applied the Mann-Whitney U test and false discovery rate (FDR) correction was used to adjust for multiple testing. Lightest-colored bars in petal plots did not reach statistical significance (defined as FDR<0.01).

FIG. 6A-F shows graphs illustrating aspects of indel burden following RT treatment and comparing indel signatures. FIG. 6A-D shows graphs depicting distributions of indel types for post-treatment mutations in the GLASS cohort, separated by RT status [FIG. 6A and FIG. 6C, RT-negative (RT−); FIG. 6B and FIG. 6D, RT-treated (RT+) and hypermutator (HM) status (FIG. 6A and FIG. 6B, HM; FIG. 6C and FIG. 6D, Non-HM)]. Patterns of indels in hypermutated samples resembled the previously identified MSI signature ID2, whereas RT-treated Non-Hypermutant gliomas harbored large similarities with ID8. Sample sizes for each subgroup are annotated. FIG. 6E shows graphs depicting a comprehensive comparison of all 17 COSMIC indel (ID) signatures in IDHmut gliomas, including absolute and relative signature contributions. The first set of graphs displays longitudinal comparisons of absolute signature contributions separated by radiation treatment status (RT− and RT+), and primary (Prim) vs. recurrence (Rec.) is evaluated for each. The second set of graphs displays longitudinal comparisons of relative signature contributions separated by radiation treatment radiation treatment status (RT− and RT+), and primary (Prim) vs. recurrence (Rec.) is evaluated for each. The paired Wilcoxon signed-rank test was applied for statistical testing for the first and second sets of graphs. The third set of graphs shows boxplots comparing absolute (upper row of panels) and relative (lower row of panels) signatures of post-treatment indels between RT-naïve (RT−) and RT-treated (RT+) samples. The Mann-Whitney U test was applied for statistical testing for the third set of graphs (ns=not significant, *=p<0.05, **=p<0.01, ***=p<0.001, ****=p<0.0001). ID8 was the only signature consistently associated with radiation therapy across different comparisons, nominating it as a robust signature of radiotherapy. FIG. 6F shows boxplots depicting the absolute and relative contributions of ID8 signature in metastatic cohort compared between cases with prior radiation treatment (RT+ pal, palliative; RT+ cur, curative) and cases without prior radiation treatment (RT−) separated by tumor types. Most tumor types showed significantly higher values of the signature in curative RT+cases. Kruskal-Wallis test was applied for statistical testing.

FIG. 7A-C presents graphs illustrating RT-associated structural variations. FIG. 7A shows association of RT with increases in large deletions and inversions in an analysis of structural variants (SVs) after RT in IDHmut glioma samples with sufficient quality for calling (n=70). For each patient, the number of SVs were calculated pre- and post-treatment. The proportion of samples with or without increase of given SVs between RT-treated (RT+) vs RT-naïve (RT−) were compared. Based on the distribution of percent increase from primary to recurrence, the cutoff was set for a >50% increase (as shown in FIG. 8A). Dark shaded bar, increase >50%; light shaded bar, increase not >50%. Statistical testing, Fisher's exact test. FIG. 7B shows proportions of IDHmut glioma samples (n=81) harboring a homozygous deletion in CDKN2A, illustrating association of RT with CDKN2A homozygous deletions. Using Fisher's exact test, proportions were compared between RT-received recurrence (RT+) vs. RT-naïve recurrence (RT−) and RT-received recurrence (RT+) vs. samples prior to treatment (Primary). (Detailed distributions of whole chromosome deletion scores are shown in FIG. 8F). FIG. 7C shows violin plots illustrating RT-associated whole chromosome aneuploidy. The upper panels show longitudinal comparisons of whole chromosome aneuploidy scores separated by RT-treatment for IDHmut glioma samples with sufficient quality for calling and complete treatment annotation (total n=69, RT-treated n=42, RT-naïve n=27). The lower panels show separation of whole chromosome aneuploidy into whole chromosome gain (left two panels) and whole chromosome loss (right two panels) scores, respectively. The increase of whole chromosome aneuploidy in RT-treated samples was associated with whole chromosome losses. Dots are proportional to the frequency of whole chromosome loss integer for each subgroup. Paired Wilcoxon rank-signed test was applied for statistical testing. FIG. 7D shows graphs illustrating validation of SV and aneuploidy results in the metastatic cohort. The upper panels show violin plots comparing whole chromosome deletion scores between RT-naïve (RT−) vs RT+ pal vs RT-cur and/or CDKN2A homdel vs. WT samples. CDKN2A homdel was associated with higher whole chromosome deletion scores, independent of RT. Within samples with CDKN2A homdel, samples that were RT-treated with curative intent showed the highest deletion scores. Dots are proportional to the frequency of whole chromosome loss integer for each subgroup. Statistical testing, Kruskal-Wallis test; detailed distributions of whole chromosome deletion scores are shown in Supp. FIG. 8G. The lower panel shows a multivariable Poisson regression model for whole chromosome deletion scores integrating RT, CDKN2A, and tumor types as variables. Curative radiotherapy and CDKN2A homozygous deletion were independently associated with higher levels of whole chromosome deletions.

FIG. 8A-G presents graphs illustrating analyses of associations of structural variants (SV) with RT and a schematic diagram. FIG. 8A shows an analysis of structural variants (SVs) in glioma samples (Translocations, Duplications, Deletions, and Inversions). For each patient, the number of SVs were calculated pre- and post-treatment and the proportional increase after therapy for each SV type was plotted separately for RT-naïve (RT−) and RT-treated (RT+) samples. Based on the distribution of proportional increase from primary to recurrence, a cutoff was defined for >50% increase that was further used for analyses (FIG. 7A). Supporting the analyses shown in FIG. 7A, FIG. 8B shows a multivariable logistic regression model fitted for the >50% increase values of the structural variant types, including radiation therapy, TMZ therapy, molecular subtype, and surgical interval as variables. Radiation therapy was independently associated with an increase in large deletions and inversions, but not duplications and translocations. FIG. 8C shows a schematic overview of separation of aneuploidy events into whole chromosome aneuploidy as a result of simple segregation errors and partial aneuploidy as a result of complex segregation errors. FIG. 8D shows violin plots of longitudinal analysis of partial aneuploidy in IDHmut glioma samples. Neither the general partial aneuploidy values (upper panels), nor the detailed separation of partial aneuploidy values into gain of chromosome arms (chromosome arm gain/neutral, lower left panels), loss of chromosome arms (chromosome arm loss/neutral, lower middle panels), and complex chromosome arm alterations (chromosome arm gain/loss, lower right panels) showed significant differences for any radiation treatment group. Dots are proportional to the frequency of whole chromosome loss integer for each subgroup. Statistical testing, paired Wilcoxon rank-signed test. FIG. 8E shows a multivariable Poisson regression model for whole chromosome losses in IDHmut glioma including molecular subtype, RT, TMZ, surgical interval, and CDKN2A status at recurrence as variables. A CDKN2A homdel, but not RT, was independently associated with higher whole chromosome losses. FIG. 8F shows density plots over integers of whole chromosome deletion scores for comparison between primary vs. recurrent glioma samples, separated by radiotherapy. In plots: primary, line with S's; recurrence, solid line. FIG. 8G shows density plots over integers of whole chromosome deletion scores for comparison between RT-naïve (RT−) vs RT+ pal vs RT+ cur and/or CDKN2A homdel vs. wild-type (WT) samples from the HMF dataset. A CDKN2A homdel was associated with higher whole chromosome deletion scores, independent of RT. Within samples with a CDKN2A homdel, samples that were RT-treated with curative intent (RT+ cur) showed the highest deletion scores. In upper graphs: CDKN2A homdel, line with X's; CDKN2A wild-type (WT), solid line. In lower graphs: lower graphs: RT−, line with asterisks; RT+ pal, solid line; RT+ cur, line with open diamonds.

FIG. 9A-B presents graphs illustrating survival probabilities for small deletion burdens. FIG. 9A shows associations of RT-related deletions with survival in the GLASS cohort. Samples were separated into three tertiles based on deletion burden at recurrence: high [(n=16) medium dark line, top tertile], intermediate [(n=16) lightest line, middle tertile], and low [(n=17, darkest line, bottom tertile]. Dotted lines indicate median overall survival times. Tertiles showed a stepwise association with survival. The left panel shows Kaplan-Meier survival plots comparing overall survival dependent on deletion burden at recurrence in RT-treated IDH mutant glioma samples (n=49 with available survival information), using the log-rank test. The middle graph shows Kaplan-Meier survival plots comparing surgical interval/time to second surgery dependent on deletion burden at recurrence using the log-rank test. The graph on the right shows Kaplan-Meier survival plots comparing post-recurrence survival dependent on deletion burden at recurrence using the log-rank test. FIG. 9B shows associations of RT-related deletions with survival in the metastatic cohort, using Kaplan-Meier survival plots comparing survival time dependent on deletion burden at metastasis in RT-treated metastases (n=958 with available survival information), using the log-rank test. Samples were separated into three tertiles based on deletion burden: high [(n=16), dark grey line with open triangles, top tertile], intermediate [(n=16) light grey line, middle tertile] and low [(n=17) dark grey line, bottom tertile). Dotted lines indicate median survival times. Tertiles showed a stepwise association with survival.

FIG. 10A-C presents graphs illustrating associations of CDKN2A status, aneuploidy burden, and ID8 burden with reduced survival. FIG. 10A shows a Kaplan-Meier survival plot (upper panel) comparing overall survival time dependent on CDKN2A status at recurrence in IDH mutant glioma samples, using the log-rank test. The lower panel shows a multivariable Cox regression model including the following variables: CDKN2A status at recurrence, TMZ treatment status, molecular subtype, and age. In plot upper panel: CDKN2A WT, dark gray line; CDKN2A homdel, dark gray line with open triangles; P<1.0e-04. FIG. 10B shows graphs comparing survival time dependent on various burdens at metastasis. The first graph shows a Kaplan-Meier survival plot comparing survival time dependent on CDKN2A status at metastasis in RT-treated metastases (n=958 with available survival information), using the log-rank test. The second graph shows a Kaplan-Meier survival plot comparing survival time dependent on aneuploidy burden at metastasis in RT-treated metastases (n=958 with available survival information), using the log-rank test. Samples were separated into three tertiles based on whole chromosome loss aneuploidy scores: high [(n=319), dark gray line with open triangles; top tertile]; intermediate [(n=319), light gray line, middle tertile], and low [(n=320), dark gray line, bottom tertile]. P=1.9e-04. The third graph shows a Kaplan-Meier survival plot comparing survival time dependent on RT signature ID8 burden at metastasis in RT-treated metastases (n=958 with available survival information), using the log-rank test. Samples were separated into three tertiles based on ID8 burden: high [(n=319), dark gray line with open triangles; top tertile], intermediate [(n=319), light gray line, middle tertile], and low [(n=320), dark gray line, bottom tertile]. P=4.0e-03. A low ID8 burden was associated with better survival, indicating a better response to RT. FIG. 10C shows a multivariable Cox regression model in RT-treated IDH mutant samples including deletion burden at recurrence as a continuous variable, and variables for CDKN2A homozygous deletion, TMZ treatment, molecular subtype, and age.

### DETAILED DESCRIPTION

Methods of the invention, in part, can be used to enhance a radiotherapy treatment of a subject with cancer. Some embodiments of the invention include contacting a cancer cell with an exogenous cytidine deaminase inhibitor (CDI) compound and the exogenous CDI compound enhances the radiosensitivity of the cancer cell. Certain embodiments of methods of the invention, the cancer cell is in a subject and a means for the contacting includes administering one or more exogenous CDI compounds to the subject. Thus, some embodiments of methods of the invention include administering an exogenous CDI compound to a subject as part of a treatment for cancer in the subject. In some embodiments of the invention, the exogenous CDI compound is part of a pharmaceutical composition, which may also include a pharmaceutically acceptable carrier. Enhanced radiosensitivity obtained using a method of the invention results can result in increased death of cancer cells in the subject, a higher efficacy of the cancer treatment, and a reduced risk of mortality for the subject receiving the exogenous CDI compound, as compared to a control. In some embodiments, a control is a level or amount of death of cancer cells, an efficacy of the cancer treatment, or a risk of mortality, respectively, in a subject or subjects not administered the exogenous CDI compound.

Studies described herein provide evidence of identification of radiotherapy-associated significant increases in the burden of small deletions (1-20 bp) and large deletions (20+bp to chromosome-arm length) in somatic DNA in subjects and cells contacted with a radiotherapy. Mutation signature analysis was performed and small deletions were characterized by a larger span size, lacking breakpoint microhomology and were genomically more dispersed when compared to pre-existing deletions and deletions in non-irradiated tumors. The results of the mutational signature analysis implicated c-NHEJ-mediated DNA damage repair and APOBEC-mutagenesis following radiotherapy. A high radiation-associated deletion burden was associated with worse clinical outcomes, and it was determined that effective repair of radiation-induced DNA damage was detrimental to patient survival. Methods of the invention are based in part on the discovery that inhibiting repair of DNA-damage that occurs as a result of radiotherapy can increase radiosensitivity in a subject thus enhancing efficacy of radiotherapy for cancer treatment.

Results and information provided herein demonstrates identification of enrichment of APOBEC associated mutational signatures and thus supports a role for inhibitors of this class of cytosine deaminases as radiosensitizers. The invention, in part, includes administration of one or more exogenous compounds that inhibit DNA repair to improve the response of cancer cells to radiotherapy. Methods of the invention include administering one or more exogenous inhibitors of radiotherapy-induced repair mechanisms, such as but not limited to an APOBEC enzyme. Methods of the invention include administering compositions comprising exogenous cytidine deaminase inhibitors to a subject to sensitize tumors in the subject to the tumor-killing effect of ionizing radiation.

### Cytidine Deaminase Inhibitors

Cytidine deamination inhibitor molecules function to inhibit activity of enzymes known as apolipoprotein B mRNA editing enzyme, catalytic polypeptide-like (APOBEC) enzymes. APOBEC enzymes deaminate deoxycytidine to deoxyuridine. Non-limiting examples of APOBEC enzymes that may be targeted for inhibited function using methods of the invention are: APOBEC1 enzyme, APOBEC3A enzyme, APOBEC3B enzyme, APOBEC3C enzyme, APOBEC3D enzyme, APOBEC3E enzyme, APOBEC3F enzyme, APOBEC3G enzyme, APOBEC3H enzyme, and an activation-induced cytidine deaminase (AID) enzyme. An APOBEC enzyme may also be referred to herein as a deaminase enzyme. The terms “inhibitor”, “inhibiting”, and “capable of inhibiting” as used herein in reference to a CDI compound and cytidine deaminase enzymes means the CDI compound reduces or stops deaminase activity of the cytidine deaminase, for example, an APOBEC enzyme. The inhibition may result from a direct interaction between the CDI compound and a deaminase enzyme or in some embodiments, may result from an indirect interaction that reduces the function, activity, and/or amount of the deaminase enzyme.

In some embodiments a CDI compound is referred to as an “exogenous” CDI compound meaning the CDI compound that is present in a cell or a subject because it is delivered from an outside source and so will be present in an amount greater than an amount that would naturally occur in the cell or subject. For example, though not intended to be limiting, a cell may naturally produce a certain level of a CDI and a method of the invention can be used to increase that level of the CDI. Any increased amount or level of a CDI that results from a method of the invention is referred to as exogenous CDI. It will be understood that an exogenous CDI may be delivered to a cell and/or administered to a subject in various forms, including but not limited to: delivery of the CDI compound, delivery of a vector encoding the CDI compound, delivery of a fusion protein encoding the CDI compound, delivery of an agent that increases expression of the CDI in a cell or subject, etc. In certain embodiments, 1, 2, 3, 4, 5, or more different exogenous CDI compounds are contacted with a cell and/or administered to a subject as part of a methods of the invention.

### Enhancement of Cancer Therapies

A subject with a cancer may be treated with a method of the invention, and/or with one or more additional therapies such as, but not limited to: surgery, radiotherapy, chemotherapy, etc. Efficacy of a radiotherapy can be enhanced using a method of the invention. As used herein the term “enhanced” means improved or increased as compared to a control efficacy of the radiotherapy in the absence or lower dosage of an exogenous CDI.

An improvement in efficacy of a radiotherapy can be determined by assessing characteristics of a cancer in a subject such as, but not limited to: progression of the cancer, regression of the cancer, spread of the cancer, an amount of cancer cell death, onset of metastatic cancer, and other art-known features of cancer. As an example, though not intended to be limiting, a subject is administered an exogenous CDI and administered a radiotherapy treatment for a cancer. The cancer is monitored following the treatments and it is determined that the cancer has regressed to a greater extent than expected based on a control level of regression in the absence of administrating the exogenous CDI. A difference in the subject's level of regression compared to a control may indicate the regression of the subject's cancer/tumor to be 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% greater than the control level of regression. In another non-limiting example, a subject is administered an exogenous CDI and a radiotherapy treatment for a cancer. The cancer is monitored following the treatments and it is determined that the cancer has progressed to a lesser extent than expected based on a control level of progression in the absence of administrating the exogenous CDI. A difference in the subject's level of progression compared to the control indicates the progression of the subject's cancer/tumor to be 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% lower/less than the control level of regression. In both cases, the difference between the results in the subject treated with a method of the invention and a control indicates the exogenous CDI enhanced the efficacy of the radiotherapy treatment of the cancer.

It will be understood that in certain embodiments of a method of the invention, contacting a cell and/or administering to a subject an exogenous CDI may increase efficacy of a chemotherapeutic cancer treatment, or other type of cancer treatment in the subject. Non-limiting examples of cancer treatments include but are not limited to: administration of a pharmaceutical agent, an immunotherapy, a modified T cell therapy, a virus-based therapy, abstention from surgery, abstention from chemotherapy, and abstention from administration of a pharmaceutical agent. In some instances, the chemotherapy and/or other cancer treatment may be used in conjunction with a radiotherapy treatment in the subject.

### Radiotherapy

A subject diagnosed with a cancer may receive one or more radiation treatments as at least a part of their cancer therapy. In some instances, a subject diagnosed with a cancer is treated with a radiation regimen that includes one or a plurality of radiation administrations to the subject as a treatment for the cancer. It will be understood that an initial radiation regimen may be followed by one or more subsequent radiation regimens if it is determined necessary for the subject. Non-limiting examples of radiation regimens for cancer in a subject are: five radiation administrations to the subject per week for three, four, five, six, seven, eight, or nine weeks; two radiation administrations per week for five weeks; and one radiation administration. It will be understood that a cancer treatment regimen may include different parameters of such as: the amount of radiation administered, the frequency of radiation administration, and the number of administrations of radiation to the subject in the treatment regimen. An initial radiation regimen may be administered to a subject following an initial identification of a cancer in the subject and a subsequent radiation regimen may be administered to the same subject following a subsequent identification in the subject.

A method of the invention can be administered to a subject before and/or during administration of a radiotherapy to the subject. In some instances a subject will be administered an exogenous CDI in advance of being administering a radiotherapy. In certain embodiments of methods of the invention a subject is administered an exogenous CDI concurrently with a radiotherapy administration. In some embodiments a subject is administered an exogenous CDI both prior to and concurrent with a radiotherapy.

The term “radiotherapy” as used herein to refer to a single administration of radiation to a subject or to refer to a regimen of two or more radiation treatments administered to a subject to treat the subject's cancer. It will be understood that the term “regimen” as used means one or a plurality of radiation treatments prescribed to a subject to treat a cancer in the subject. For example, upon a diagnosis of a cancer in a subject, a healthcare professional may prescribe a regimen of one or more radiation administrations to treat the cancer in the subject.

It will be understood that radiotherapy may be delivered using various art-known means or forms, non-limiting examples of which are: external beam radiation and brachytherapy. The term radiotherapy may be used herein in reference to palliative radiotherapy and in reference to a curative radiotherapy. In some embodiments, a radiotherapy administered to a subject is a palliative radiotherapy. It will be understood that the term “palliative radiotherapy” means a radiation therapy administered to a subject to do one or more of: shrink a cancer; shrink a tumor; slow a cancer's growth; slow a tumor's growth; stop or slow progression of a cancer or tumor; and stop, slow, or reduce symptoms caused by the cancer or tumor. Palliative radiotherapy may be administered to a subject to reduce focal symptoms of advanced cancer, either symptoms arising from a primary tumor or one or more metastatic growths in the subject. In some embodiments, palliative radiotherapy comprises administration of high energy X-rays to a focused region in the subject, for example a tumor site in the subject. In certain embodiments, a radiotherapy administered to a subject is a curative radiotherapy. A curative radiotherapy may include radiation administered to a subject that is one or more of: more broadly administered to the subject, more frequently administered to the subject, and at a higher dose level of radiation delivered to a subject, compared to a palliative radiotherapy. A subject may be administered one or more different forms and may be administered 1, 2, 3, 4, 5, 6, 7, different administrations of a radiotherapy.

### Effective Amounts for Treatments

A CDI compound is administered to a subject in an effective amount for enhancing treatment of a cancer. An “effective amount” is an amount necessary or sufficient to realize a desired biologic effect. For example, an effective amount of a compound of the invention could be that amount necessary to (i) slow or halt progression of the cancer; or (ii) reverse one or more symptoms of the cancer. According to some aspects of the invention, an effective amount is that amount of a CDI compound, which when administered to a subject enhances a therapeutic response in a cancer in the subject. The biologic effect may be the amelioration and or absolute elimination of symptoms resulting from the cancer. In another embodiment, the biologic effect is the complete abrogation of the cancer, as evidenced for example, by a diagnostic test that indicates the subject is free of the cancer.

Typically an effective amount of a CDI compound will be determined in clinical trials, establishing an effective dose for a test population versus a control population in a blind study. In some embodiments, an effective amount will be an amount that results in a desired response, e.g., an amount that diminishes a cancer in cells or tissues in a subject with the cancer. Thus, an effective amount to treat a cancer, may be the amount that when administered decreases activity of a cytidine deaminase enzyme (such as, but not limited to an APOBEC enzyme) in the subject to an amount that that is lower than the amount that would occur in the subject or tissue without the administration of the exogenous CDI compound. In the case of treating a cancer the desired response may be reducing or eliminating one or more symptoms of the cancer in a cell and/or subject. The reduction or elimination may be temporary or may be permanent. The status of the cancer can be monitored using routine diagnostic methods for cancers. In some aspects of the invention, a desired response to treatment of the cancer can be delaying the onset or even preventing the onset of the cancer in the subject.

An effective amount of a CDI compound (also referred to as a “pharmaceutical compound” may also be determined by assessing physiological effects of administration on a cell or subject, such as a decrease of a cancer and/or a decrease in cytidine deamination following administration. Assays suitable to determine efficacy of a pharmaceutical compound of the invention will be known to those skilled in the art and can be employed for measuring the level of the response to a treatment and an amount of a pharmaceutical compound administered to a subject can be modified based, at least in part, on such measurements. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon a characteristic of a cancer being treated. As a non-limiting example an effective amount of an exogenous CDI compound may be higher when used to treat a cancer that exhibits a higher level of cytidine deaminase enzymes and/or cytidine deaminase enzyme activity compared with an amount when used to treat a cancer that exhibits a lower level of cytidine deaminase enzymes and/or cytidine deaminase activity.

The effective amount of a compound of the invention to treat a cancer may vary depending upon the specific CDI compound used, the mode of delivery of the CDI compound, and whether it is used alone or in combination. The effective amount for any particular application can also vary depending on such factors as the type and stage of the cancer being treated, the particular CDI compound being administered, the size of the subject, or the severity of the cancer. A skilled artisan can empirically determine the effective amount of a particular compound of the invention without necessitating undue experimentation. Combined with the teachings provided herein, an effective prophylactic or therapeutic treatment regimen can be planned which does not cause substantial toxicity and yet is effective to treat the particular subject.

A pharmaceutical compound dosage may be adjusted by an individual health care provider or veterinarian, particularly in the event of any complication. A therapeutically effective amount typically varies from 0.01 mg/kg to about 1000 mg/kg, from about 0.1 mg/kg to about 200 mg/kg, or from about 0.2 mg/kg to about 20 mg/kg, in one or more dose administrations daily, for one or more days. The absolute amount will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Multiple doses of CDI compounds of the invention are also contemplated. In some instances, a CDI compound of the invention, can be administered at least daily, every other day, weekly, every other week, monthly, etc. Doses may be administered once per day or more than once per day, for example, 2, 3, 4, 5, or more times in one 24 hour period.

Pharmaceutical compounds of the invention may be administered alone, in combination with each other, and/or in combination with other cancer therapies, or other treatment regimens that are administered to subjects with a cancer. Pharmaceutical compositions used in the foregoing methods preferably are sterile and contain an effective amount of a therapeutic compound that will enhance efficacy of a cancer treatment, such as a radiotherapy to a level sufficient to produce the desired response in a unit of weight or volume suitable for administration to a subject.

The doses of a CDI compound to enhance a radiotherapy can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Administration Methods

A variety of administration routes for a CDI compound are available. The particular delivery mode selected will depend, of course, upon the particular condition being treated and the dosage required for therapeutic efficacy. Methods of this invention, generally speaking, may be practiced using any mode of administration that is medically acceptable, meaning any mode that produces effective levels of protection without causing clinically unacceptable adverse effects. In some embodiments of the invention, a compound of the invention may be administered via an oral, enteral, mucosal, percutaneous, and/or parenteral route. The term “parenteral” includes subcutaneous, intravenous, intramuscular, intraperitoneal, and intrasternal injection, or infusion techniques. Other routes include but are not limited to nasal (e.g., via a gastro-nasal tube), dermal, vaginal, rectal, and sublingual. Delivery routes of the invention may include intrathecal, intraventricular, or intracranial. In some embodiments of the invention, a compound of the invention may be placed within a slow release matrix and administered by placement of the matrix in the subject. In some aspects of the invention, a CDI compound may be delivered to a subject cell using nanoparticles coated with a delivery agent that targets a specific cell or organelle.

CDI compounds of the invention may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. According to methods of the invention, the compound may be administered in a pharmaceutical composition. In general, a pharmaceutical composition comprises the compound of the invention and a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the compound such as a CDI compound to enhance treatment of the cancer.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compounds of the invention may be administered directly to a tissue. In some embodiments, the tissue to which the compound is administered is a tissue in which the cancer is likely to arise. Direct tissue administration may be achieved by direct injection. Compounds may be administered once, or alternatively they may be administered in a plurality of administrations. If administered multiple times, the compounds may be administered via different routes. For example, the first (or the first few) administrations may be made directly into the affected tissue while later administrations may be systemic.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with or without an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of compounds.

In yet other embodiments, a delivery vehicle is a biocompatible microparticle or implant that is suitable for implantation into the mammalian recipient. Exemplary bioerodible implants that are useful in accordance with this method are described in PCT Publication No. WO 95/24929 (incorporated by reference herein), which describes a biocompatible, biodegradable polymeric matrix for containing a biological macromolecule. Such delivery means are well known in the art and can be used to achieve sustained release of a compound of the invention in a subject, and may be selected not to degrade, but rather, to release by diffusion over an extended period of time.

Both non-biodegradable and biodegradable polymeric matrices can be used to deliver the compounds of the invention to the subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, compounds of the invention may be delivered using the bioerodible implant by way of diffusion, or by degradation of the polymeric matrix. Exemplary synthetic polymers for such use are well known in the art. Biodegradable polymers and non-biodegradable polymers can be used for delivery of compounds of the invention using art-known methods. Bioadhesive polymers such as bioerodible hydrogels (see H. S. Sawhney, C. P. Pathak and J. A. Hubell in Macromolecules, 1993, 26, 581-587, the teachings of which are incorporated herein) may also be used to deliver compounds of the invention for treatment. Additional suitable delivery systems can include time-release, delayed release or sustained release delivery systems. Such systems can avoid repeated administrations of the compound, increasing convenience to the subject and the physician. Many types of release delivery systems are available and known to those of ordinary skill in the art. (See for example: U.S. Pat. Nos. 5,075,109; 4,452,775; 4,675,189; 5,736, 152; 3,854,480; 5,133,974; and 5,407,686 (the teaching of each of which is incorporated herein by reference). In addition, pump-based hardware delivery systems can be used, some of which are adapted for implantation.

Therapeutic formulations of compounds of the invention may be prepared for storage by mixing the compound having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

### Controls

In some embodiments of the invention, methods may include comparing a treatment of a cancer that includes administration of an exogenous CDI compound. Results of such treatments may be compared to control results. As used herein a “control” may be a normal control or a control known to have been treated with a different dose of an administered CDI compound or other cancer therapeutic. A control may be obtained from historical databases of efficacy of radiotherapy or other cancer treatment in the absence of administration of an endogenous CDI compound. Means of selecting and using appropriate controls in comparative, diagnostic, treatment, and assay methods are well known in the art. In some embodiments of the invention, a control is based on an assessment of a cancer in a subject prior to being administered a treatment method of the invention. Thus, in some embodiments of the invention, a control for a subject reflects a baseline cancer status or symptoms of the subject prior to treatment and changes in one or more symptoms or characteristics of the cancer in the subject before and after treatment that includes administration of an exogenous CDI compound can be assessed.

Assessing symptoms and/or characteristics of a cancer in a subject to determine a change, non-limiting examples of which are: progression, regression, metastatic spread, tumor growth, etc. are well-known in the art and can be applied in conjunction with methods described herein.

### Subject, Diseases, Cells, and Samples

As used herein, a subject shall mean a vertebrate animal including but not limited to a human, mouse, rat, guinea pig, rabbit, cow, dog, cat, horse, goat, and primate, e.g., monkey. In certain aspects of the invention, a subject may be a domesticated animal, a wild animal, or an agricultural animal. Thus, the invention can be used to test for and treat diseases or conditions in human and non-human subjects. For instance, methods and compositions of the invention can be used in veterinary applications as well as in human treatment regimens. In some embodiments of the invention, the subject is a human. In some embodiments of the invention, a subject has a cancer.

A subject at risk of having or suspected of having a cancer is a subject who has been diagnosed with a cancer or is believed likely to have a cancer based on factors such as clinical examination, symptoms, and other art-known methods to assess cancers. For example, though not intended to be limiting, visual and/or physical examination of a subject may suggest the subject as likely to have a cancer. Art-known diagnostic procedures and assessments can be used to determine if a subject is a risk of having, is believed to have, is likely to have, or is diagnosed as having a cancer. Methods of the invention can be used to treat a cancer. As used herein the term “treat” means reduce the cancer, slow down growth and/or spread of a cancer, reducing or eliminating symptoms of a cancer, increasing death of cancer cells, etc.

Non-limiting examples of cancers that can be treated using an embodiment of a method of the invention are: a brain cancer, a neuroblastoma, a glioma, a bone cancer, a soft-tissue cancer, a breast cancer, a colon cancer, a rectal cancer, an esophageal cancer, a head and neck cancer, a lung cancer, an ovarian cancer, a prostate cancer, a skin cancer, a urinary tract cancer, a central nervous system (CNS) cancer, and a uterine cancer. In some embodiments of the invention a brain cancer is a glioma. In some embodiments, a subject has a cancer that exhibits elevated cytidine deaminase activity.

In some embodiments of a method of the invention, a cancer that is treated is a cancer exhibiting elevated cytidine deaminase activity and/or APOBEC enzyme activity as compared to a control cytidine deaminase activity and/or APOBEC enzyme activity, respectively. It will be understood that the term “elevated” used in regarding to APOBEC enzyme or cytidine deaminase activity means the activity is higher when compared to the control cytidine deaminase activity and/or APOBEC enzyme activity. Non-limiting examples of a control cytidine deaminase activity or APOBEC enzyme activity include: a level of cytidine deaminase activity or APOBEC enzyme activity, respectively, in a non-cancer cell or tissue and a level of cytidine deaminase activity and/or APOBEC enzyme activity respectively, in a cell of a different cancer type or different tumor type.

Cells that can treated using methods and compounds of the invention include but are not limited to mammalian cells, human cells, vertebrate cells, non-human mammalian cells, cultured cells, tumor cells, somatic cells, etc.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

Radiotherapy Treatment is Associated with an Increased Small Deletion Burden

### Materials and Methods

The methods described herein apply to Example 1 and subsequent Examples as appropriate.

### Patient Cohort

A cohort of 190 patients with high-quality longitudinal DNA sequencing data was curated, including treatment naïve primary and matched post-treatment first recurrence tumor samples from the GLASS dataset [Barthel, F. P. et al., Nature 576, 112-120 (2019)]. Paired samples were classified into three subtypes according to the 2016 World Health Organization (WHO) classification: IDH mutant with 1p/19q co-deletion (IDHmut-codel), IDH mutant without 1p/19q co-deletion (IDHmut-noncodel), and IDH wild type (IDHwt) [Louis, D. N. et al., Acta Neuropathol. 131, 803-20 (2016)]. The GLASS cohort used herein consisted of n=106 whole genome sequencing (WGS) samples (n=53 primary samples, n=53 matched first recurrence samples) and n=274 whole exome sequencing (WES) samples (n=106 primary samples, n=106 matched first recurrence samples). Detailed information on sequence platforms, capture kits, and read length information were as previously described [Barthel, F. P. et al., Nature 576, 112-120 (2019)].

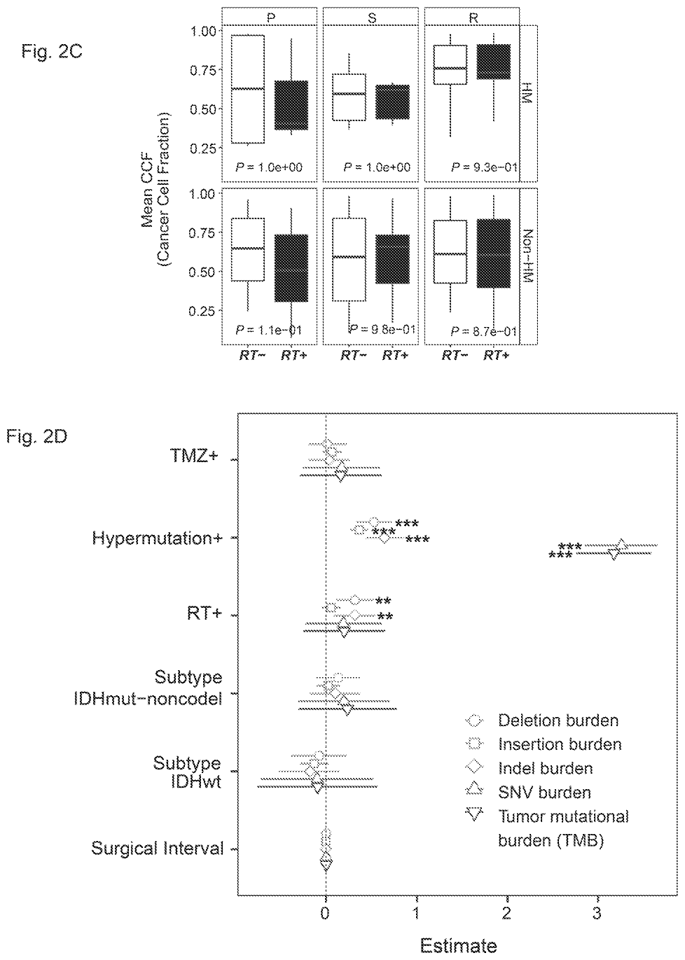

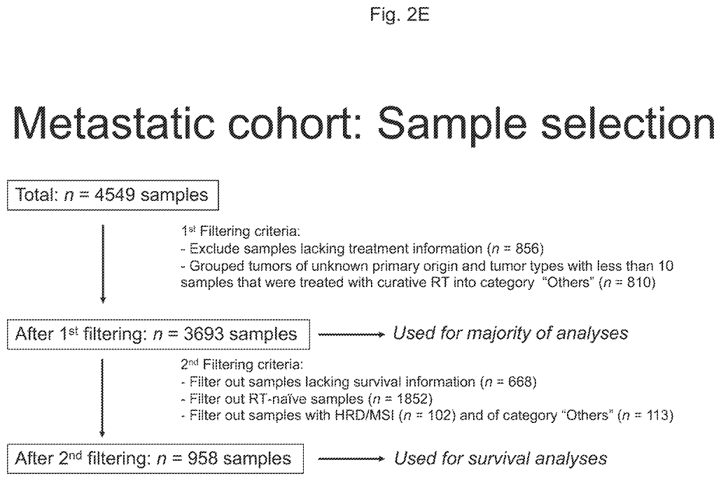

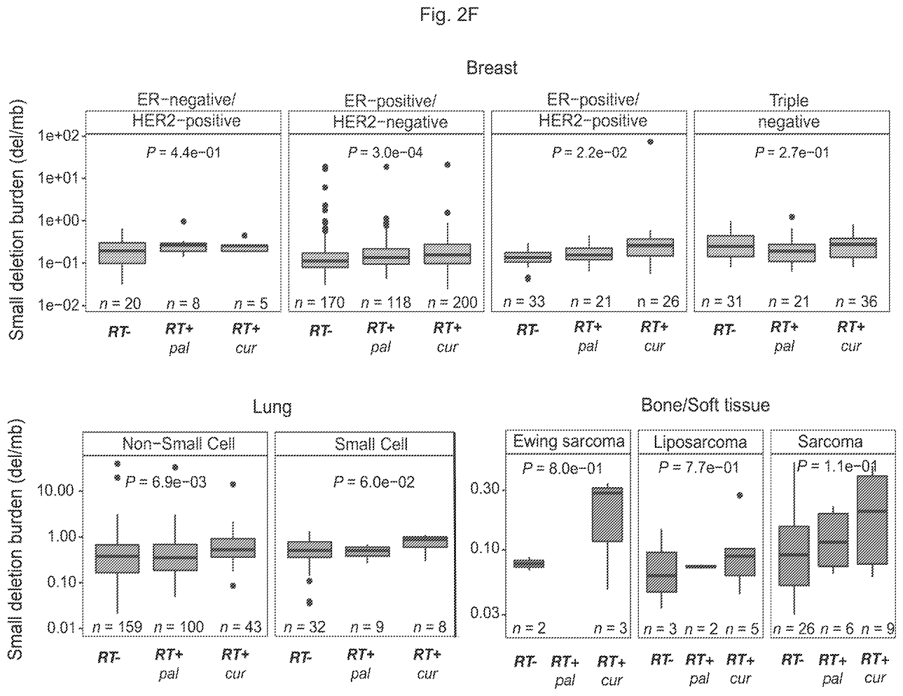

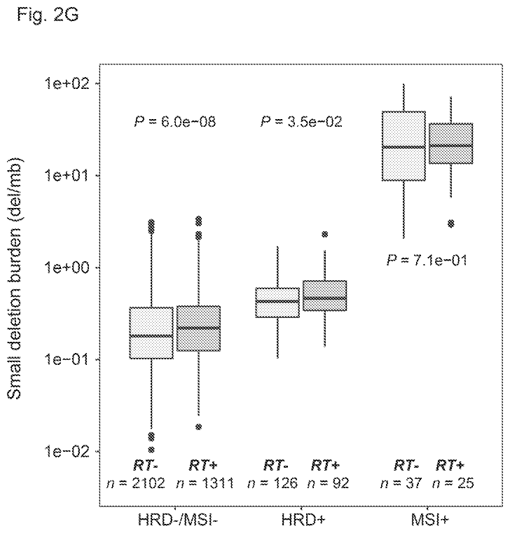

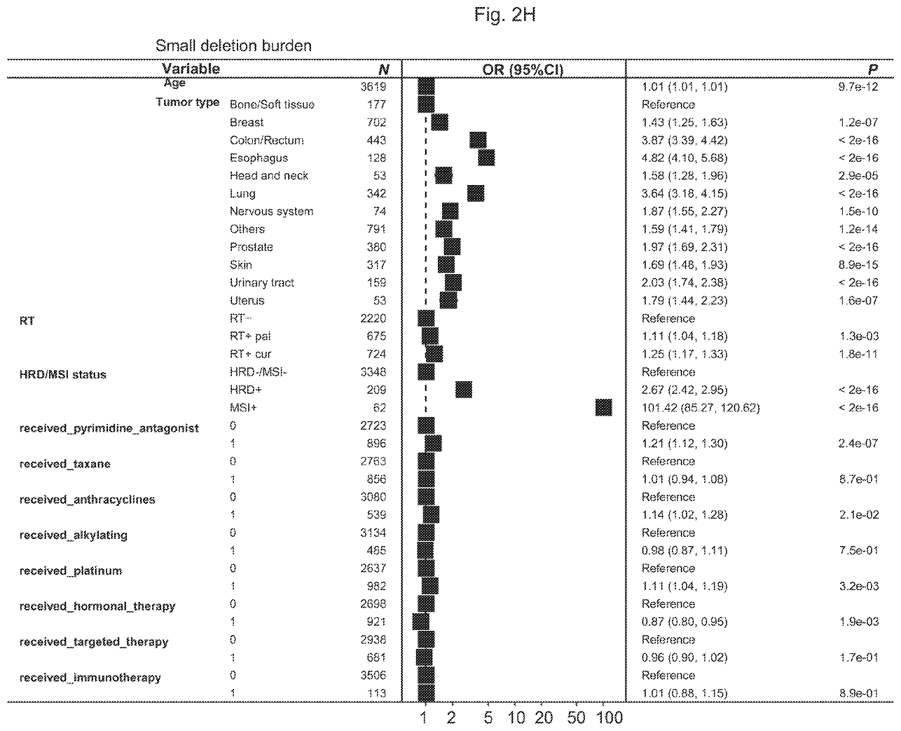

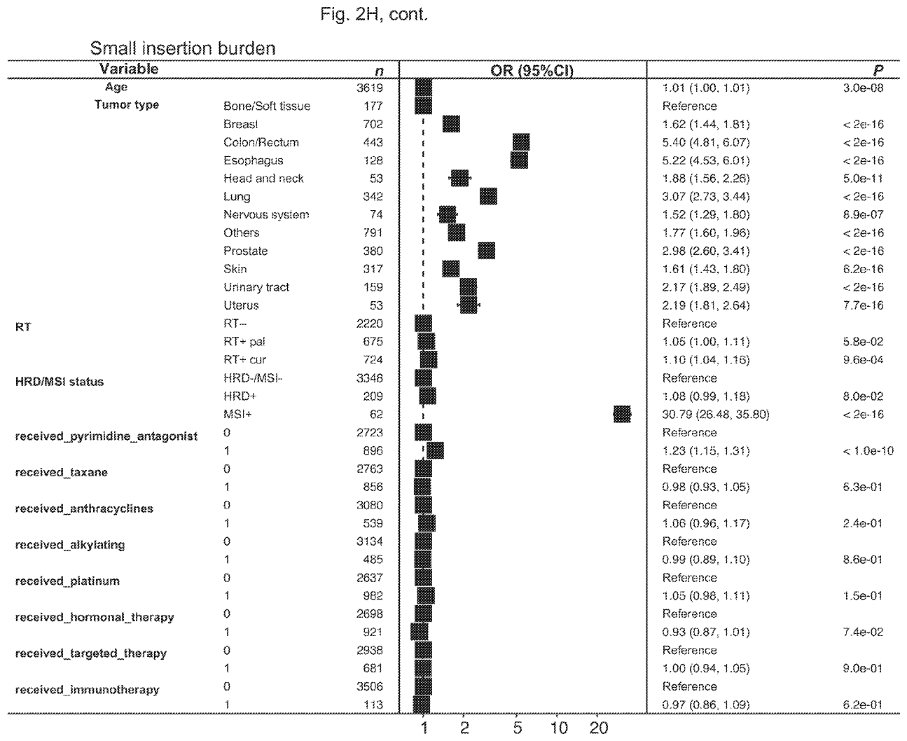

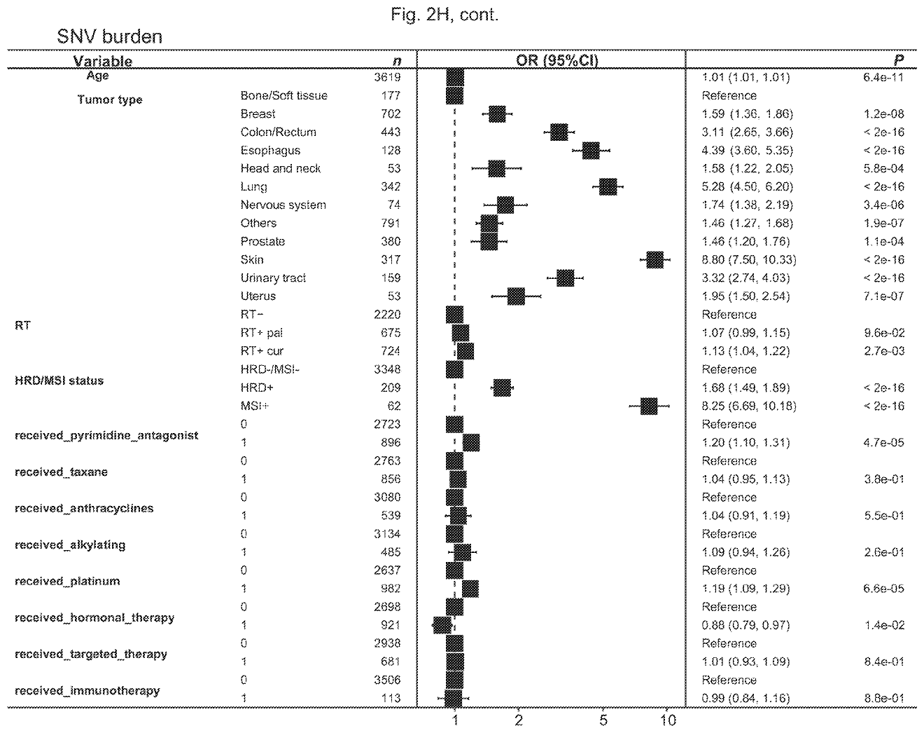

For validation analyses, a metastatic cohort from the Hartwig Medical Foundation (HMF; Amsterdam, NL) was curated, comprising a total of 4,549 samples [Priestley, P. et al., Nature 575, 210-216 (2019)]. The HMF cohort consisted of metastatic tumor samples collected following local or systemic treatment as part of the CPCT-02 (NCT01855477) and DRUP (NCT02925234) clinical trials. Biopsy samples from a wide range of tumor types collected at various hospitals across the Netherlands were sequenced at the core facilities of the Hartwig Medical Foundation. Whole genome sequencing (WGS) was performed for each sample according to standardized protocols [Bins, S. et al., Oncologist 22, 33-40 (2017)]. Detailed information on sequence platforms, capture kits, and read length information were as previously described [Priestley, P. et al., Nature 575, 210-216 (2019)]. VCF files with mutations and associated metadata were downloaded from The Hartwig Medical Database (database.hartwigmedicalfoundation.nl). After application of filtering criteria (as described in FIG. 2E) a set of n=3,693 samples were defined and used for the majority of analyses described below herein. For survival analyses, curative RT-treated samples with sufficient survival information (n=958) were selected. All prior radiotherapy data were extracted using clinical data as present in the CPCT-02 eCRF on Dec. 8, 2020. These data were not cleaned and represented the data entered by the clinical sites. The prior radiotherapy was categorized as curative intent, palliative intent, or other. All other instances were manually curated. All adjuvant/neo-adjuvant or post-operative radiotherapy was considered curative intent radiotherapy. All local radiotherapy for pain relief or other symptom-directed goals were considered as palliative. Some items were not specified, and those events were not included in the analysis. All radiotherapy for non-malignant disease states was excluded, specifically for gynecomastia treatment after castration. Over- or underrepresentation of the radiation signatures could not be excluded as it was not known whether the metastases that were biopsied were not already present at the time of radiotherapy.

### Variant Calling

Variant calling in the GLASS dataset was performed according to the GATK Best practices using GATK 4.1.0.0 as previously described [Barthel, F. P. et al., Nature 576, 112-120 (2019)]. Briefly, GATK 4.1.0.0 was used for variant calling in tumor samples against a matched normal control. Additionally, panels of normals were constructed across multiple control samples from the same tissue source and sequencing center. Variants were broadly filtered for germline variants, cross-sample contamination, read orientation, and sequence context. Variants were called across all samples for a given patient. Variants with a minimum coverage of 10 reads in both primary and recurrence and a minimum VAF of 10% for either the primary or the recurrence were included for further analysis. Variants were considered to be present if at least one mutant read was detected in a sample. Mutations directly overlapping with known repeat regions according to the repeatmasker database were removed. Specifically, all variants in known repeat regions were filtered out, including DNA satellites, microsatellites, long terminal repeats, transposable elements (LINE/SINE elements), and low complexity regions. Variant clonality was inferred for each patient individually using PyClone (v.0.13.1) and as previously described [Barthel, F. P. et al., Nature 576, 112-120 (2019)]. Pipeline scripts can be found at github.com/fpbarthel/GLASS.

### Mutation Burden Comparison

The mutation burden was calculated as the number of mutations per megabase (Mb) with at least 10× coverage and stratified by variant type. The overall tumor mutation burden (TMB) was calculated as the sum of the burden of small deletions, small insertions, and single nucleotide variants. Recurrent tumors with greater than 10 mutations per Mb were considered hypermutated as previously described [Barthel, F. P. et al., Nature 576, 112-120 (2019)]. For the comparison of mutation burden between RT-treatment groups in the GLASS dataset, the burden of mutations unique to the recurrent tumors and therefore acquired after treatment was calculated. To adjust for confounding covariables, a multivariable log-linear regression model was fitted using the glm function in R. In addition to RT-treatment, TMZ-treatment, hypermutation, surgical interval in months, and molecular subtype were included as variables. The small deletion burden in the GLASS dataset was not confounded by batch effects. Accordingly, the full therapy and tumor type information was included for mutation burden analyses in the Hartwig metastatic cohort. To adjust for negative infinite values resulting from the log-transformation in the GLASS cohort, a constant value of 1 was added to the log function. For the metastatic cohort, the log-transformation did not result in (negative) infinite values and therefore did not necessitate the addition of a constant value.

### Statistical Methods

All data analyses were conducted in R 3.6.1 (broadly using tidyverse 1.3.0), Python 3.7.3, and PostgreSQL 10.5. R was interfaced with the PostgreSQL database used for data storage using the unixODBC 2.3.6 driver plus DBI 1.0.0 and odbc 1.1.6 R packages. All survival analyses including Kaplan-Meier plots and Cox proportional hazards models were conducted using the R packages survival and survminer. For unpaired group comparisons the Mann-Whitney U test and Kruskal-Wallis test were used, and for paired longitudinal comparisons the Wilcoxon signed-rank test was applied. Forest plots were generated using the R package forestmodel. Survival times for the GLASS dataset were calculated as described previously [Barthel, F. P. et al., Nature 576, 112-120 (2019)]. In the HMF metastatic cohort, survival was calculated starting from the date of biopsy to date of death. For patients that were alive, the last date of follow-up (date of treatment end) was used as censoring.

### Data and Code Availability

Processed sequencing data from the GLASS project used in this and subsequent Examples are available at synapse.org/glass. Processed sequencing data from the Hartwig Medical Foundation (HMF) dataset used in this and subsequent Examples are available at hartwigmedicalfoundation.nl. The repeatmasker database used in this and subsequent Examples is available at repeatmasker.org/. Pipeline scripts used in this and subsequent Examples are available at github.com/fpbarthel/GLASS. Custom scripts for analyses performed in this and subsequent Examples are available at github.com/TheJacksonLaboratory/Radiation_Scars.

### Results

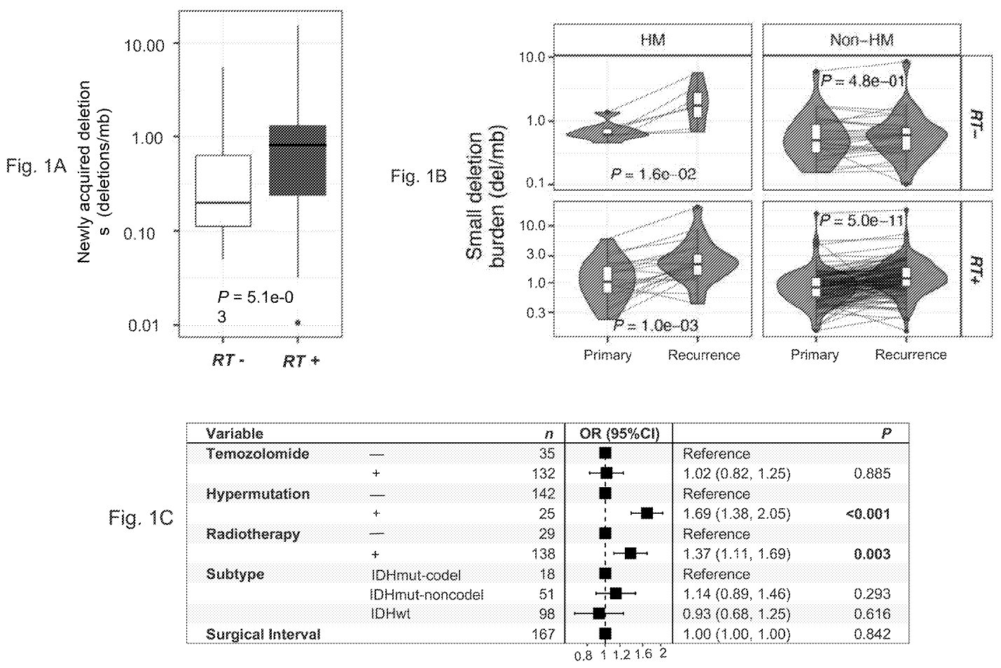

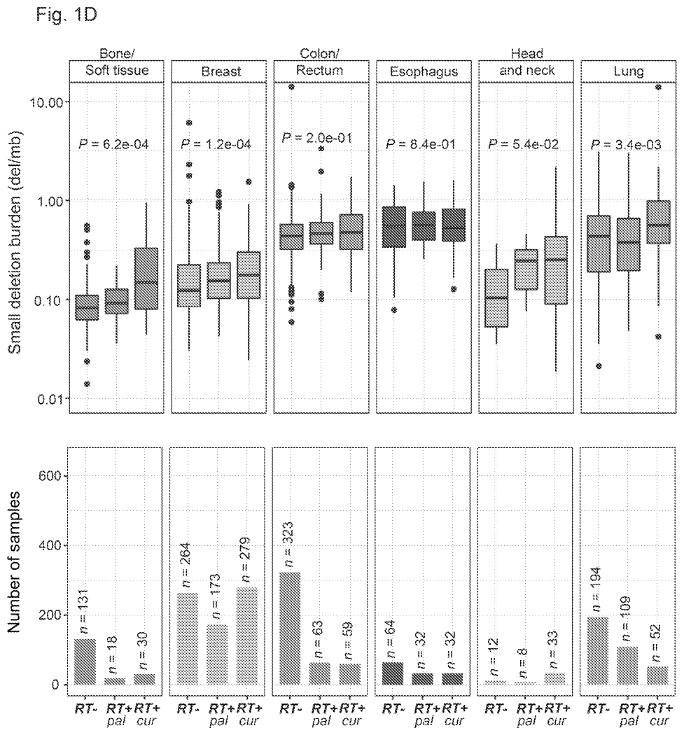

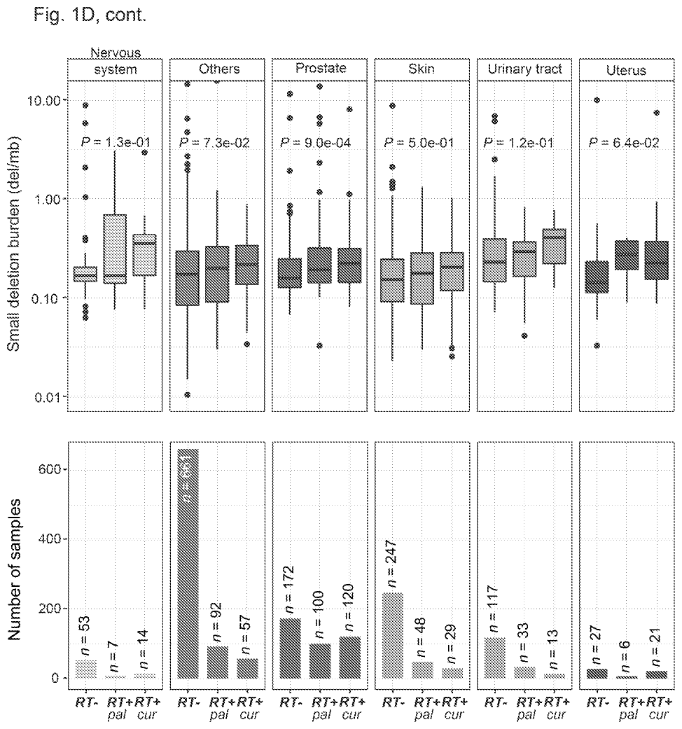

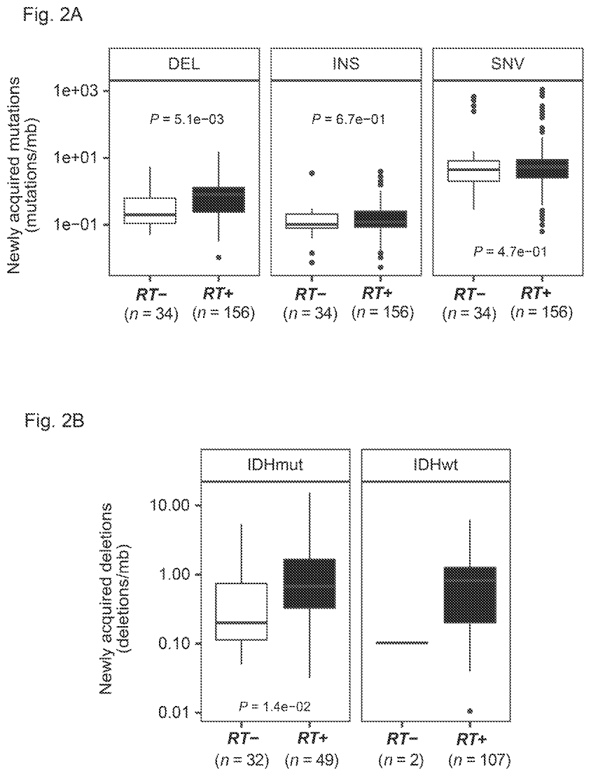

First, the contributions of radiotherapy (RT) and temozolomide (TMZ) on the burden of somatic mutations including small insertions/deletions (indels, length of 1 to 20 bp) and somatic single nucleotide variants (sSNVs) in the exomes of matched pre- and post-treatment glioma samples (n=190) were analyzed, of whom 119 (63%) cases had received the combination of RT and TMZ, 19 (10%) cases had received RT alone, 13 (7%) cases underwent TMZ treatment, and 16 (8%) cases had received no RT or TMZ treatment. For 23/190 (12%) cases, TMZ annotation was lacking, 18 of which had received RT. For each patient, mutations were separated into pre- (present in the initial tumor) and post-treatment (only present in the recurrent tumor). The mutation burden (average mutation frequency per megabase (Mb)) of post-treatment mutations was then calculated. A median of 0.68 new small deletions/Mb were acquired in recurrent RT-treated glioma which was significantly higher than the median of 0.19 new small deletions/Mb acquired in recurrent RT-naïve gliomas (RT−; FIG. 1A, P=5.1e-03, Mann-Whitney U test), and significantly higher than the small deletion burden detected at diagnosis (FIG. 1B). RT was not associated with a significant increase in the sSNV burden (FIG. 2A, P=4.7e-01, Mann-Whitney (test) or small insertion burden (FIG. 2A, P=6.7e-01, Mann-Whitney U test). The increase in small deletions was particularly pronounced in the subset of gliomas marked by the presence of mutations in IDH1 or IDH2, a clinically relevant subtype [Louis, D. N. et al., Acta Neuropathol. 131, 803-20 (2016)] predominantly consisting of grade II and III gliomas (FIG. 2B, P=1.4e-02, Mann-Whitney U test), while the number of RT-naive recurrent cases among IDH wild-type glioma was too small to test for differences (n=2, vs n=107 RT-treated cases). To ensure that these changes were not primarily due to TMZ-associated hypermutation (tumor mutation burden exceeding 10 mut/Mb at recurrence) [Barthel, F. P. et al., Nature 576, 112-120 (2019)], the cohort was stratified by hypermutation status. Hypermutation was associated with an increase in small deletions independent of RT-treatment, whereas amongst non-hypermutators only tumors from patients that received RT showed a significant increase in small deletions (FIG. 1B, P=5.0e-11, paired Wilcoxon signed-rank test), further implicating the observed increase in small deletions as a potential consequence of RT. To evaluate the independence of this finding from potential confounders, a log-linear regression model was fitted that included TMZ-treatment, glioma molecular subtype, time interval between surgeries, and hypermutation. RT was independently associated with an increase in small deletions (FIG. 1C, P=3e-03, t-test), directly attributing the observed increase in small deletions to RT-treatment. Cancer cell fractions were determined and it was found that post-treatment deletions in RT-treated patients did not show clonality differences compared to post-treatment deletions in RT-naïve patients, suggesting that these deletions were not more clonal/subclonal (FIG. 2C, hypermutant: P=9.3e-01, non-hypermutant: P=8.7e-01, Mann-Whitney U test). Comparing the pre-treatment mutation burden and aneuploidy scores between glioma patients that acquired a high burden versus a low burden of post-treatment deletions revealed no significant differences, suggesting that these genomic characteristics of the pre-RT tumor are not predictive of the acquisition of small deletions in response to radiotherapy.

Importantly, 30% (41/136) of non-hypermutant samples gained more than 1 del/Mb following radiotherapy, whereas only 7% (2/27) of RT-naïve non-hypermutators acquired more than 1 del/Mb (P=1.6e-02, Fisher's exact test). Among the samples treated with ionizing radiation, 35% (55/156) showed a doubling of the small deletion burden when compared with the primary tumor. The effect of RT on mutational burden was significant for small deletions and not significant for other types of somatic mutations such as insertions and sSNVs (FIG. 2D). Conversely, TMZ-associated hypermutation was associated with significant increases in the burden of all types of mutations (FIG. 2D).

Following these observations, it was hypothesized that radiotherapy may similarly increase the number of small deletions in other tumor types. To test this hypothesis, whole-genome sequencing-derived mutational profiles were evaluated from 3693 metastatic tumors with complete treatment annotation (as described in FIG. 2E), available via the Hartwig Medical Foundation (hereafter “HMF” or “Hartwig”) dataset [Priestley, P. et al., Nature 575(7781), 210-216 (2019)]. Tumors were separated by site of origin and the small deletion burdens of RT-treated and naïve tumors were compared. RT-treated tumors were further stratified depending on whether the treatment intent was curative (RT+ cur, n=739) or palliative (RT+ pal, n=689), which generally differ in the applied cumulative dosage of ionizing radiation [Lutz, S. T. et al., J Clin. Oncol. 32, 2913-2919 (2014)]. While this analysis was restricted to single time-point mutational profiles, a significantly higher small deletion burden associated with curative RT was observed in multiple tumor types, including bone/soft tissue (RT-cur: median 0.15 del/Mb, RT−: median 0.08 del/Mb, P=6.2e-04, Kruskal-Wallis test), lung (RT+ cur: median 0.56 del/Mb, RT−: median 0.43 del/Mb, P=3.4e-03, Kruskal-Wallis test), and breast (RT+ cur: median 0.18 del/Mb, RT−: median 0.12 del/Mb, P=1.2e-04, Kruskal-Wallis test) cancers (FIG. 1D). Further separation into tumor subtypes revealed that the observed patterns were present in both lung cancer types (FIG. 2F, Non-small cell lung cancer: P=6.9e-03 and small cell lung cancer: P=6.0e-0.2, Kruskal-Wallis test), but in breast cancer these were restricted to ER-positive subtypes (FIG. 2F, ER-positive/HER2-negative: P=3.0e-04 and ER-positive/HER2-positive: P=2.2e-02, Kruskal-Wallis test). Tumors treated palliatively with RT frequently presented an intermediate state in between the RT- and RT+ cur cohorts, suggesting an association between RT-treatment derived small deletion burden and RT dose exposure.

DNA repair deficient tumors had been shown to harbor an increased mutational load [Campbell, B. B. et al., Cell 171, 1042-1056.e10. (2017)]. To determine whether a DNA repair defective background had an impact on the small deletion burden in the HMF dataset, information on microsatellite instability (MSI) and homologous recombination deficiency (HRD) in the HMF dataset was derived from previous data [Nguyen, L. et al., Nat. Commun. 11, 5584 (2020)]. Notably, HRD+ and particularly MSI+tumors harbored significantly more small deletions compared to samples that were HRD−/MSI− (FIG. 2G, P<2.2e-16, Kruskal-Wallis test). RT-treatment was associated with an increase in small deletion burden in HRD−/MSI− (FIG. 2G, P=6.0e-08, Mann-Whitney U test) and HRD+tumors (P=3.5e-02), but not in MSI+tumors (P=7.1e-01). These results raised the possibility that DNA repair deficiencies like HRD and MSI confounded the association between RT-treatment and the small deletion burden. To address this, HRD and MSI status were included in the multivariable log-linear regression analysis, which showed that RT-treatment was associated with an increase in the small deletion burden independent of a number of potential confounders, including a DNA repair defective background (FIG. 2H).

The next assessment was whether the small deletion burden was associated with mutations in selected genes (ATM, ATR, CHEK1, CHEK2, PARP1, PRKDC, TP53 and WEE1) involved in the DNA damage response (DDR). This analysis indicated that DDR mutations were universally associated with a significantly higher small deletion burden. Log-linear regression was used to correct for potential confounding variables, including age, tumor type, DNA damage repair background, DRR gene mutations, and various cytotoxic treatment regimens (e.g. taxane, platinum, anthracyclines, alkylating agents) that have been previously associated with increased mutation burdens [Pich, O. et al., Nat. Genet. 51, 1732-1740 (2019)]. Results from this analysis revealed a robust association between both palliative and curative radiotherapy treatment but not any other therapy and small deletions (FIG. 2H, RT+ cur vs. RT-naïve: odds ratio=1.25, P<1e-03, t-test), confirming that the increased small deletion burden associated with radiotherapy was independent of tumor type, HRD, MSI, DDR gene mutations, or additional cytotoxic therapy.

To verify the causal association between RT and acquired small deletions, a previously published dataset [Kucab et al., Cell 177, 821-836.e16. (2019)] consisting of whole-genome sequencing data from 324 human-induced pluripotent stem cells (iPSCs) exposed to known or suspected environmental carcinogens, including two iPSCs treated with ionizing radiation, was re-analyzed. The small deletion burden was found to be significantly higher in the RT-treated iPSCs compared to controls (FIG. 2I, P=2.0e-02, Mann-Whitney U test). In contrast, no significant difference in small insertion burden was observed (P=1.8e-01). Strikingly, the ionizing radiation group showed the highest median burden of small deletions across all treatment modalities, further substantiating our human tissue analysis (FIG. 2J).

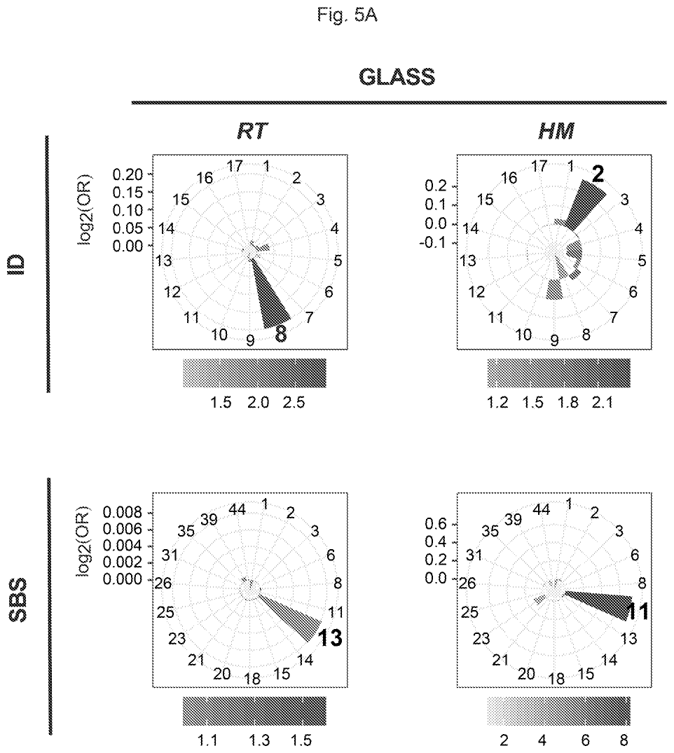

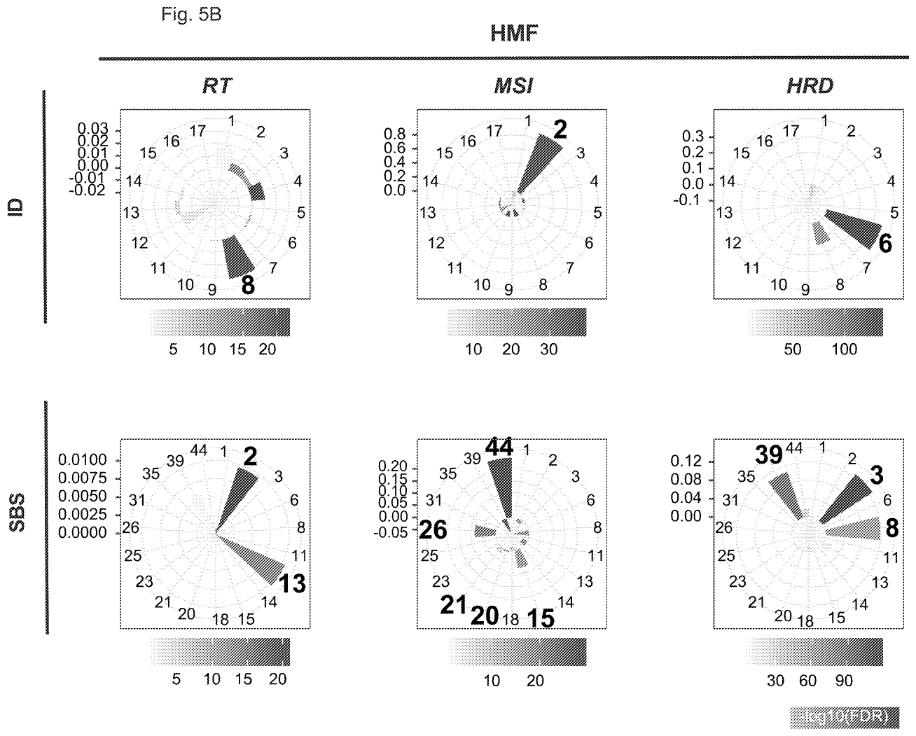

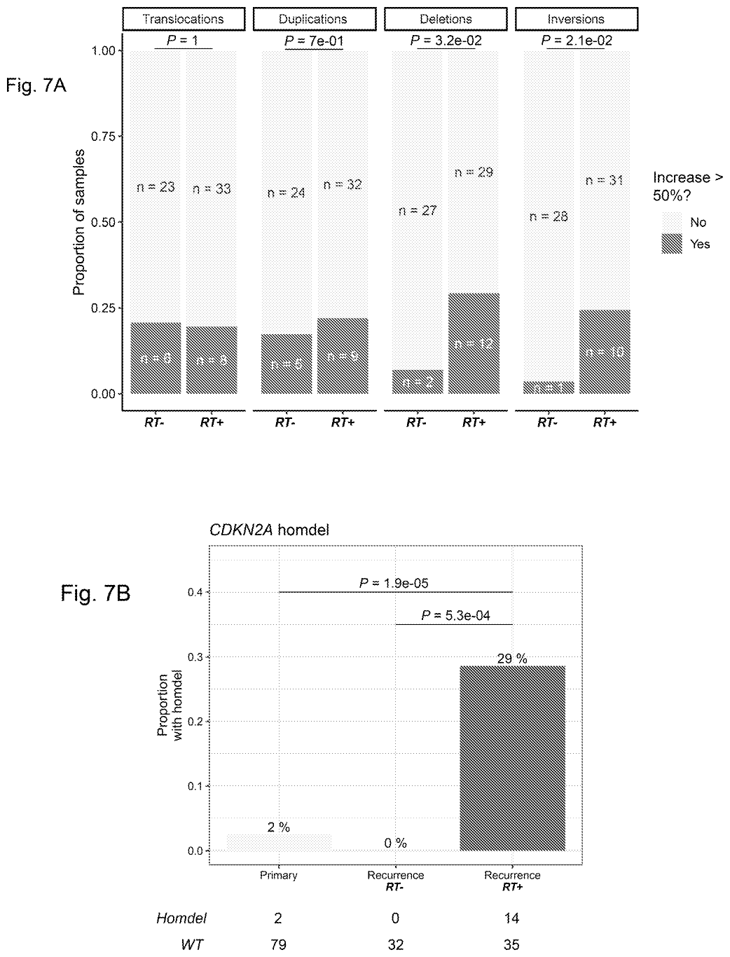

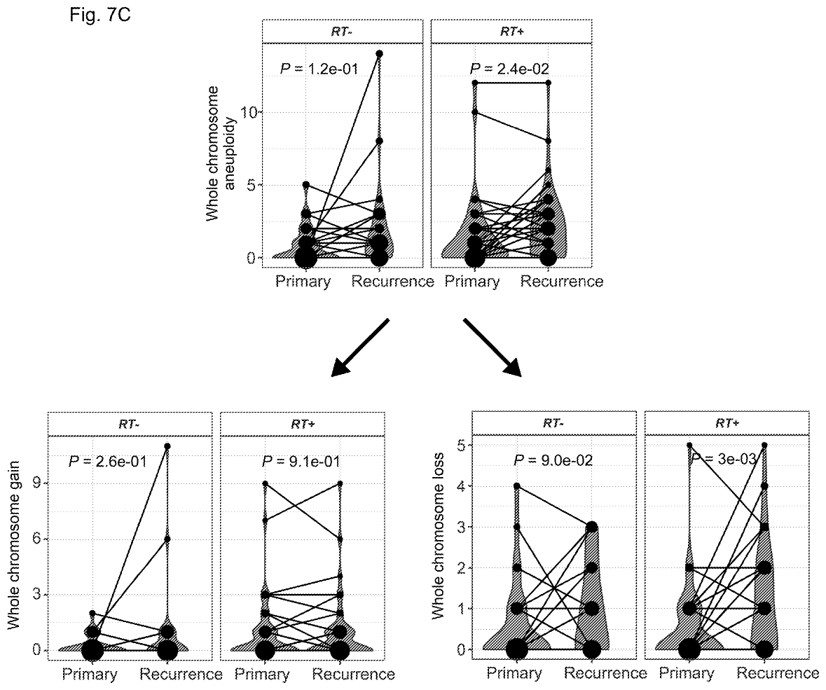

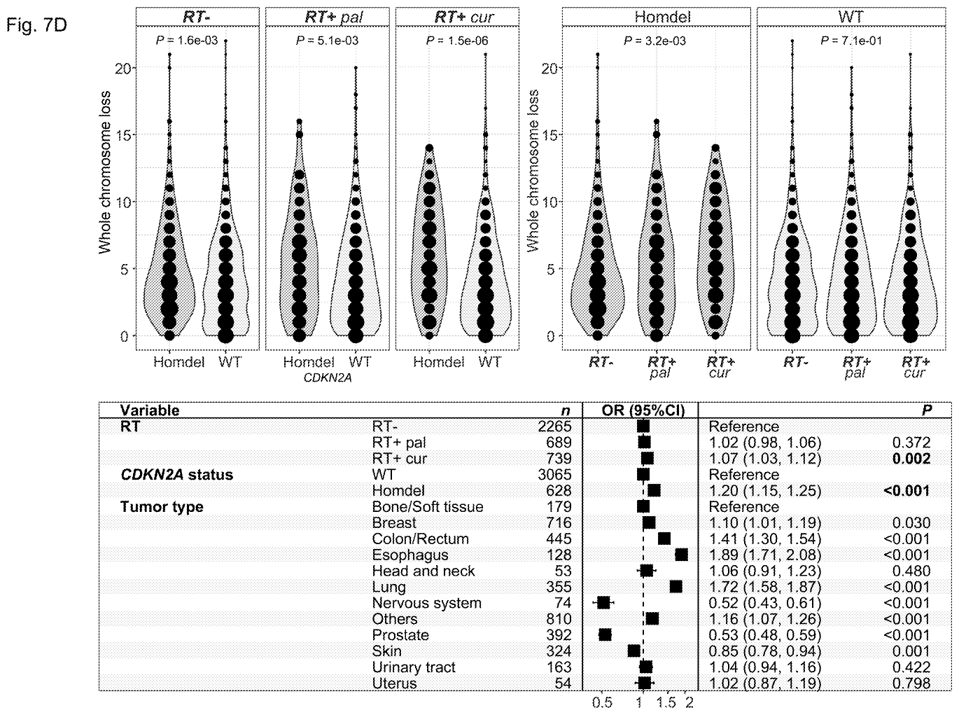

Pre- and post-treatment small deletions were compared to previously defined mutational indel signatures [Alexandrov, L. B., et al., Nature 578, 94-101 (2020)]. Indels from the GLASS cohort were separated into three fractions: private to primary (P, pre-treatment), shared between primary and recurrence (S, pre-treatment) and private to recurrence (R, post-treatment). For each fraction, the contribution of indel signatures was calculated and mean contributions between RT-treated and RT-naïve samples were compared. Indel signature 8 (ID8) had the highest contribution in the recurrence-only fraction (mean contribution=0.22) and was significantly higher in tumors from patients that received RT (P=1.68e-4, Q)=8.56e-3, Mann-Whitney U test and false discovery rate, respectively). Furthermore, in RT-treated patients but not RT-naïve patients (FIG. 7E) comparing ID8 values before and after treatment revealed significant increases in absolute (P=4.5e-07, Paired Wilcoxon rank-signed test) and relative (P=0.0023) ID8 contributions, respectively. Specifically, in the post-treatment fraction RT was significantly associated with ID8 (absolute ID8: P=2.3e-05, relative ID8: P=7.4e-05). Signature ID8 is composed of ≥ 5 bp deletions without microhomology and has previously been linked to DSB repair by c-NHEJ, providing further evidence that radiation-induced DSBs are primarily repaired via c-NHEJ. A previous analysis had indicated an increase in the proportion and frequency of ID12 signature mutations in the Hartwig dataset [Pich, O., et al., Nat. Genet. 51, 1732-1740 (2019)]. Directed by the instant findings in the GLASS cohort, an increase in ID8 mutations, not ID12, was the strongest and most significant association with radiotherapy treatment among metastatic tumors (FIG. 5A-B). Both absolute and relative ID8 values are significantly higher in RT-treated samples when compared to RT-naïve samples, and this association was independent of tumor type (FIG. 7F, FIG. 7G).

### Example 2

### Radiotherapy-Associated Small Deletions Harbor a Characteristic Genomic Signature

### Materials and Methods

Materials and methods used were as described in Example 1 and below herein, as applicable.

Association of Deletions with Non-B DNA Structures

The genomic locations of non-canonical DNA structures were derived from the Non-B DNA database [Cer, R. Z. et al., Nucleic Acids Res. 41, D94-D100 (2013)]. For every variant position and, for comparison, for 250,000 randomly sampled positions from the reference genome, the distance to non-B features was calculated as a continuous (absolute distance to genomic feature in bp) or categorical (position in or up to 100 bp to genomic feature—yes/no) value. A Mann-Whitney U test was used for differences in the genomic properties of variants in radiation-induced and non-radiation-induced tumors after adjusting for random background distribution.

dNdScv

For quantification of selection processes at the level of individual genes dependent on radiation therapy, dN/dS ratios were calculated as previously described [Barthel, F. P. et al., Nature 576, 112-120 (2019)]. Briefly, the R package dNdScv [Martincorena, I. et al., Cell 171, 1029-1041.e21 (2017)] was run using the default and recommended parameters for each mutational fraction (private to primary, shared between primary and recurrence, and private to recurrence). All analyses were conducted separately within radiotherapy-naïve and radiotherapy-treated groups.

### Sequence Microhomology

Sequence microhomology was determined by iteratively comparing the 3′ end of the deleted sequence to the 5′ flanking sequence. Any deletion demonstrating at least 2 nt of homology was considered microhomology-mediated. The homologous sequence was characterized and further analyzed for the presence of 1 nt, 2 nt, and 3 nt repeats. The repeat unit and number of repeats were quantified.

### Results

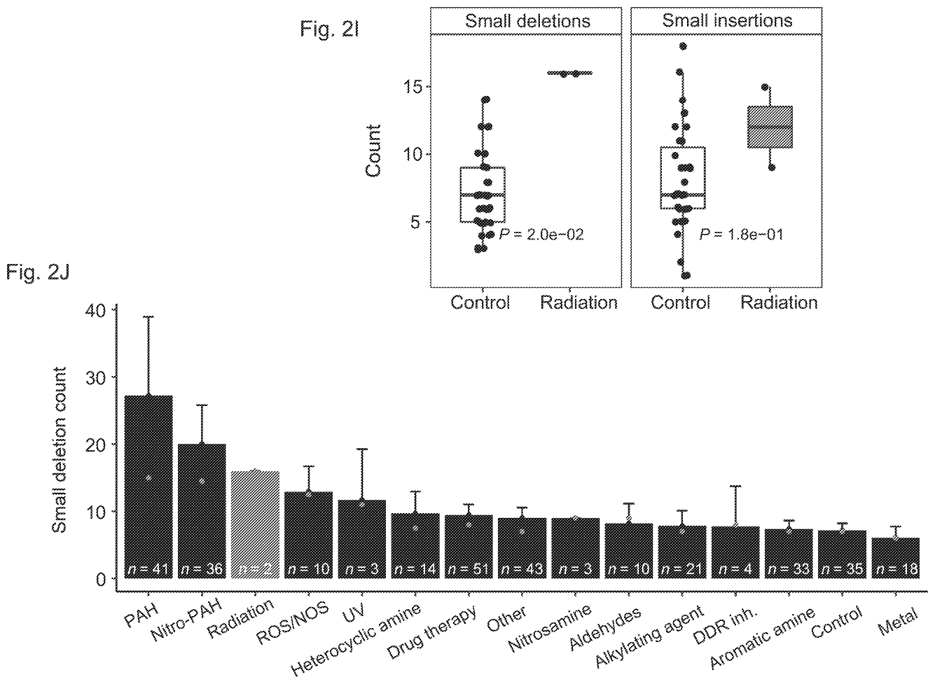

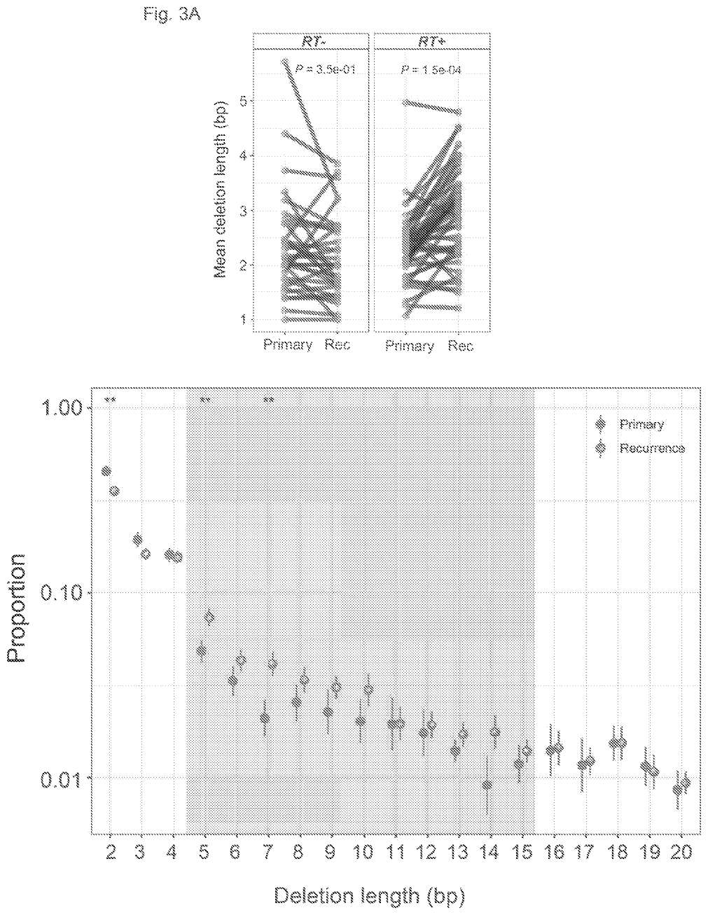

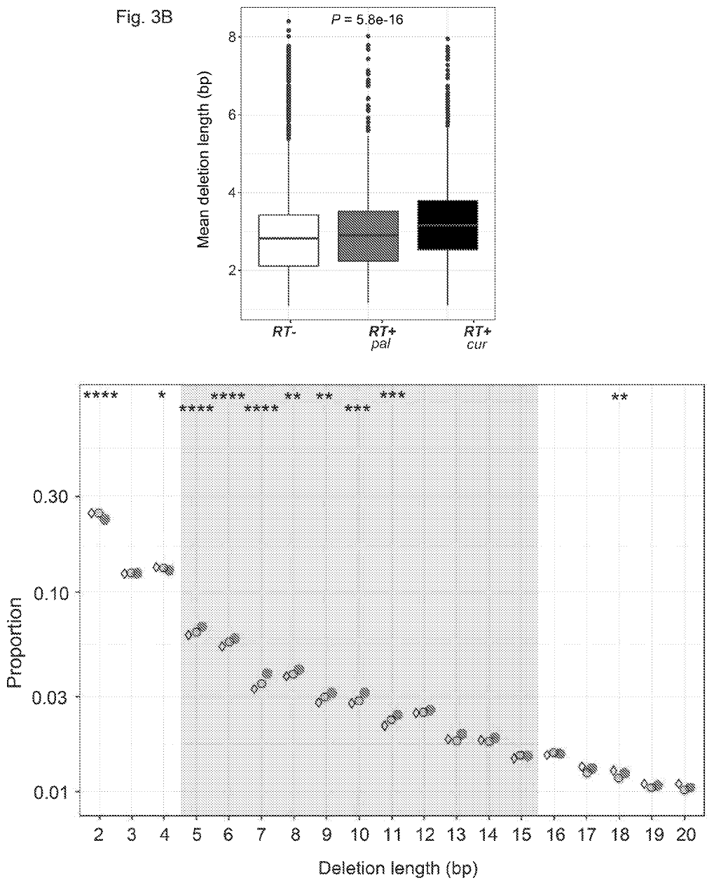

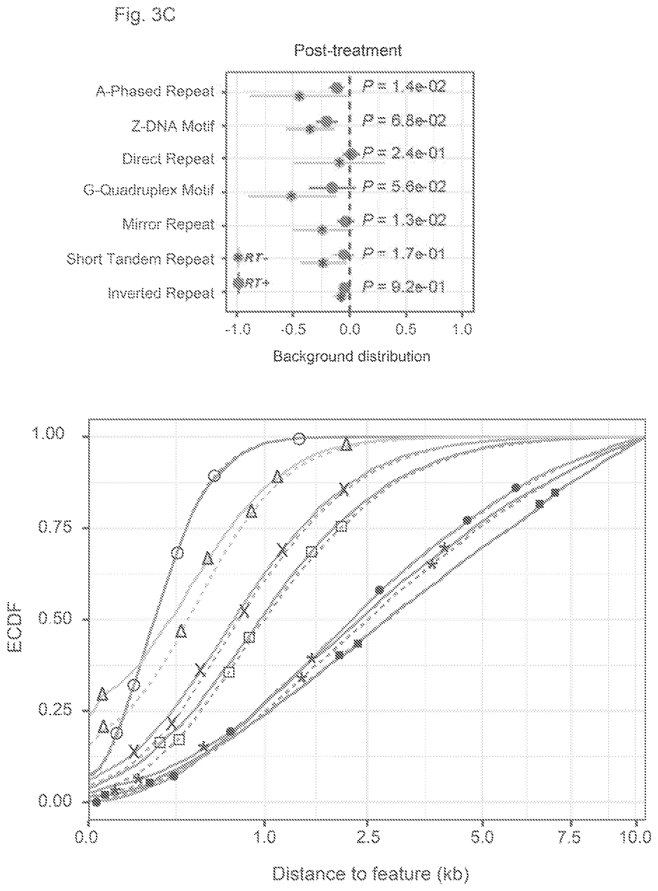

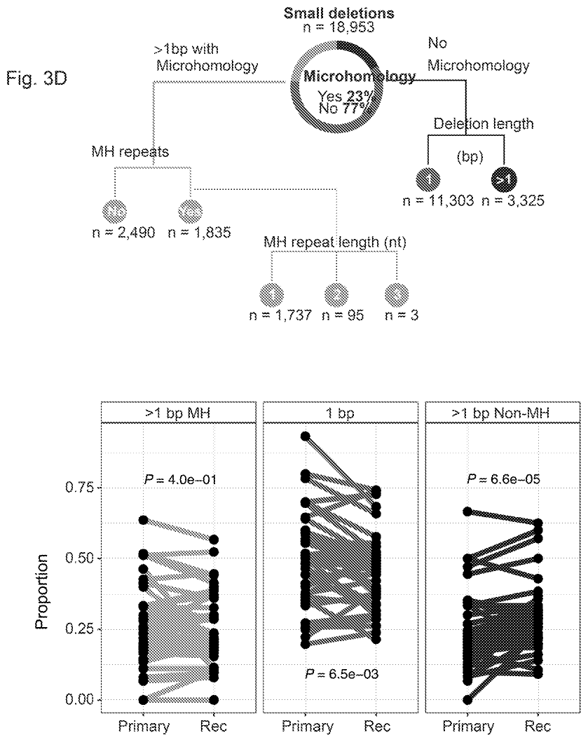

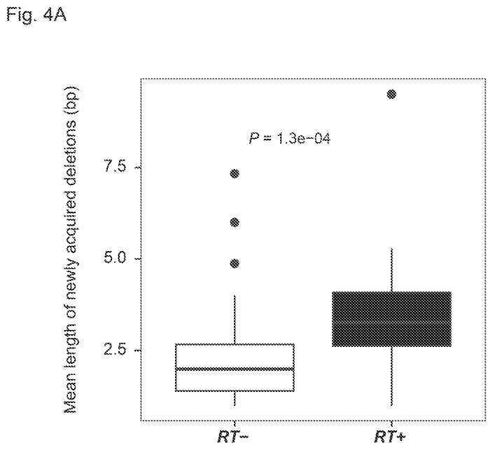

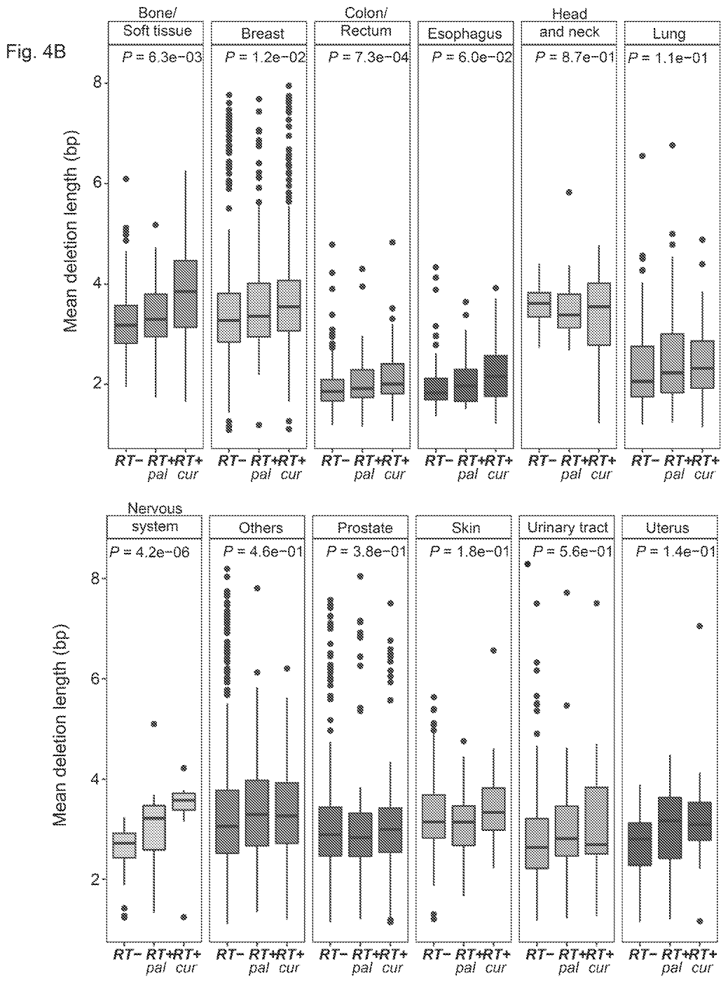

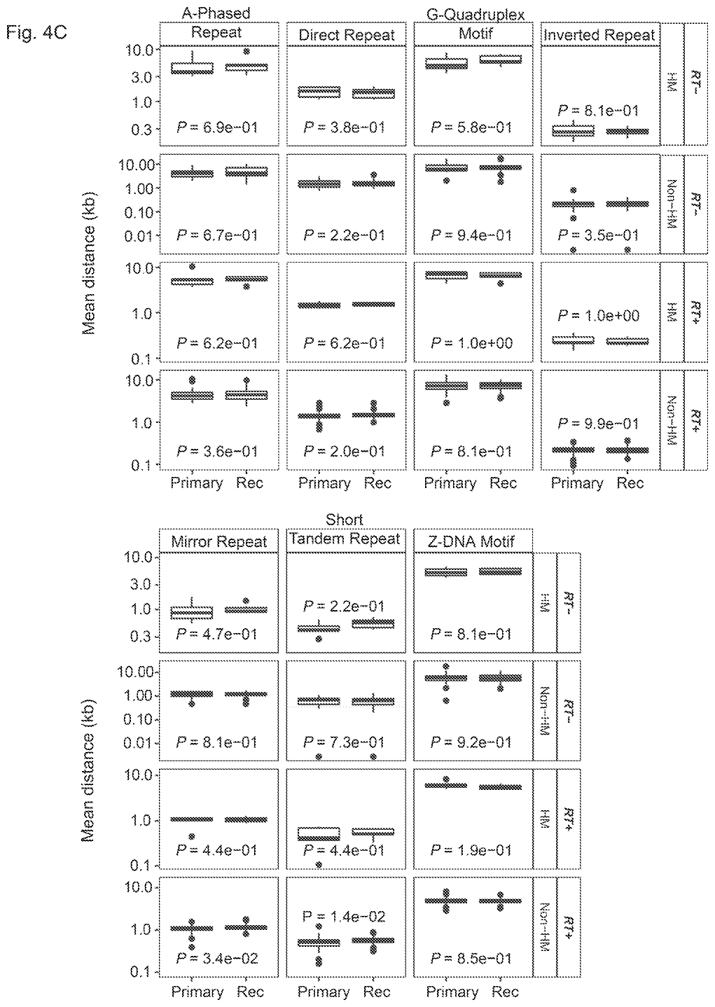

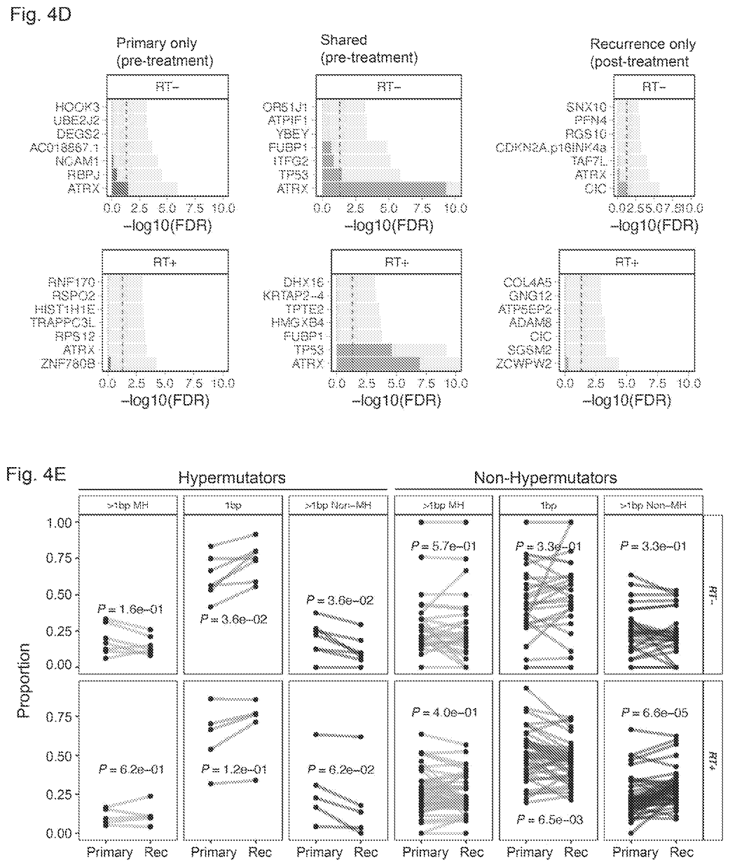

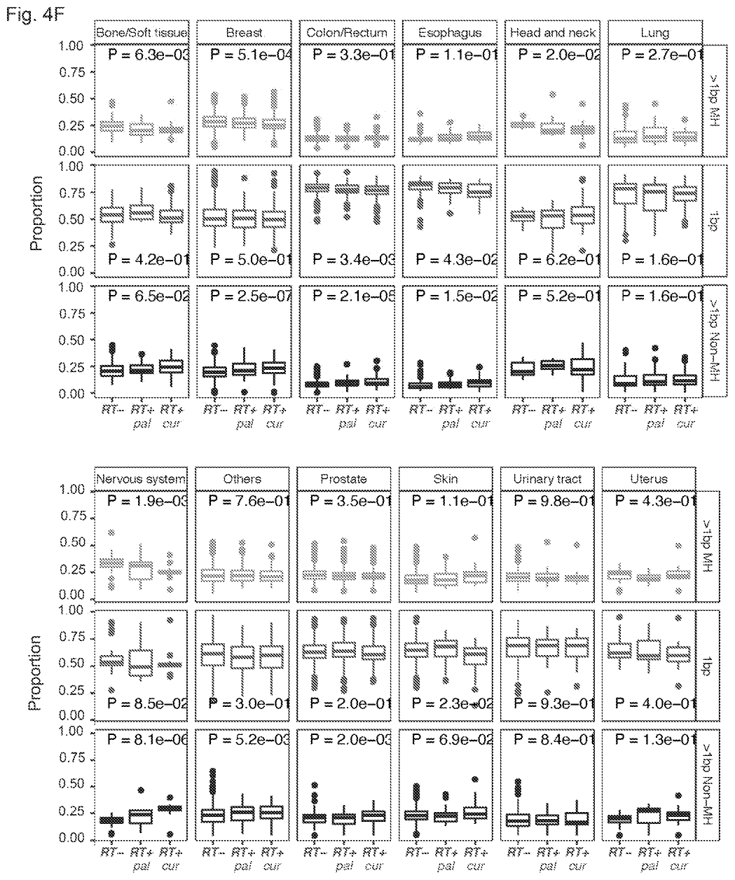

Characteristics of RT-associated small deletions, such as length distribution and breakpoint microhomology, may be able to provide insights on their etiology. Such features were explored in the GLASS dataset, limiting the analysis to IDH mutant gliomas (RT+, n=49; RT−, n=32) due to the imbalance in radiation treatment amongst IDH wild-type gliomas in the GLASS cohort (RT+, n=107 vs RT−, n=2). Small deletions in recurrent tumor samples that were treated with RT showed increased deletion lengths (FIG. 3A, upper panels, RT+: P=1.5e-04; RT−: P=3.5e-01, paired Wilcoxon signed-rank test). This increase was particularly associated with new deletions that occurred after therapy (FIG. 4A, P=1.3e-04, Mann-Whitney (test), supporting the idea that RT leads to longer deletion lengths (FIG. 3A, upper panels). Moreover, a detailed analysis of the size distribution of deletions revealed a shift towards deletions of length ˜5-15 bp following RT-treatment in non-hypermutated gliomas (FIG. 3A, lower panel).

Comparing RT-treated and RT-naïve metastatic tumor samples from the single time-point HMF dataset showed a similar larger average deletion length for both palliative and curative RT-treated tumor samples (FIG. 3B, FIG. 4B). Moreover, a shift was also observed in deletion span from small 1-4 bp deletions towards medium-sized 5-15 bp deletions (FIG. 3B). A stepwise increase in deletion length was observed for palliative and curative RT-treatment, respectively, providing further evidence for a dose and exposure association. Taken together, these results indicated not only an increased deletion burden, but also highlighted distinct characteristics of RT-associated small deletions.

B-DNA is the common right-handed, double helical formation of DNA. Non-canonical non-B-DNA structures and fragile repeat-rich DNA may be more prone to acquiring mutations [Georgakopoulos-Soares, I. et al., Genome Res. 28, 1264-1271 (2018)]. Therefore, it was hypothesized that RT-induced deletions were more likely to occur in these fragile regions of the genome. The link between small deletions and these genomic features was investigated by adjusting for a random background distribution. Importantly, deletions following RT showed less variability and higher similarity to the random background distribution compared to non-RT-induced deletions (FIG. 3C, upper). Furthermore, comparison of GLASS pre- and post-treatment deletions indicated that deletions following radiotherapy showed larger distances to non-B DNA features (FIG. 3C, lower, FIG. 4C). The lack of or reduced association between radiation-induced deletions and the analyzed genomic features, such as repeats and G-quadruplex motifs, suggested that ionizing radiation associated small deletions occurred in a largely stochastic manner, occurring independently from the intrinsic mutagenicity of the fragile regions of the genome analyzed.

It was assessed whether RT-associated small deletions showed enrichment in driver genes. The covariate-adjusted normalized ratio between non-synonymous and synonymous mutations (dN/dS) was computed in order to identify selection of mutations at the level of individual genes separately for GLASS pre- and post-treatment fractions (FIG. 4D) [Martincorena, I. et al., Cell 171, 1029-1041.e21 (2017)]. Genes with dN/dS ratios strongly deviating from one were thought to be under selection and may be associated with the RT+small deletion phenotype. No evidence was found for significant selection for any genes in the post-treatment fraction following radiation therapy. Because these analyses focused on acquired deletions only present after RT, they could not also be performed in the HMF set where pre-treatment samples were unavailable. The results in IDH-mutant glioma further supported the notion that RT-associated deletions did not occur at particular genomic loci, instead showing a more dispersed genomic spread.

Small deletions can be the result of error-prone DSB-repair utilizing mechanisms such as c-NHEJ and a-EJ/MMEJ [Chang, H. H. Y. et al, Nat. Rev. Mol. Cell Biol. 18, 495-506 (2017)]. To investigate whether a preference for DSB-repair pathway choice following RT existed, microhomology sequences were computed at breakpoints and deletions were characterized based on size, microhomology, and repeat content (FIG. 3D, FIG. 2E). Deletions without microhomology comprised the majority of deletions in the dataset (77%, FIG. 3D). However, in non-hypermutant gliomas receiving ionizing radiation a significant increase in >1 bp deletions without microhomology was observed (FIG. 3D, P=6.6e-05, Paired Wilcoxon signed rank test) and conversely a decrease in 1 bp-deletions was observed (FIG. 3D, P=6.5e-03, Paired Wilcoxon signed-rank test). Using the same three categories described in FIG. 3D, comparison of RT-treated and RT-naïve metastatic tumors from the HMF dataset demonstrated comparable results (FIG. 4F). These data suggested that c-NHEJ was the preferred pathway for repairing radiation-induced DNA damage.

### Example 3

Distinct ID and SBS Mutational Signatures Associated with Radiotherapy

### Materials and Methods

Materials and methods were as described in Examples 1 and 2 above and as below herein, as appropriate.

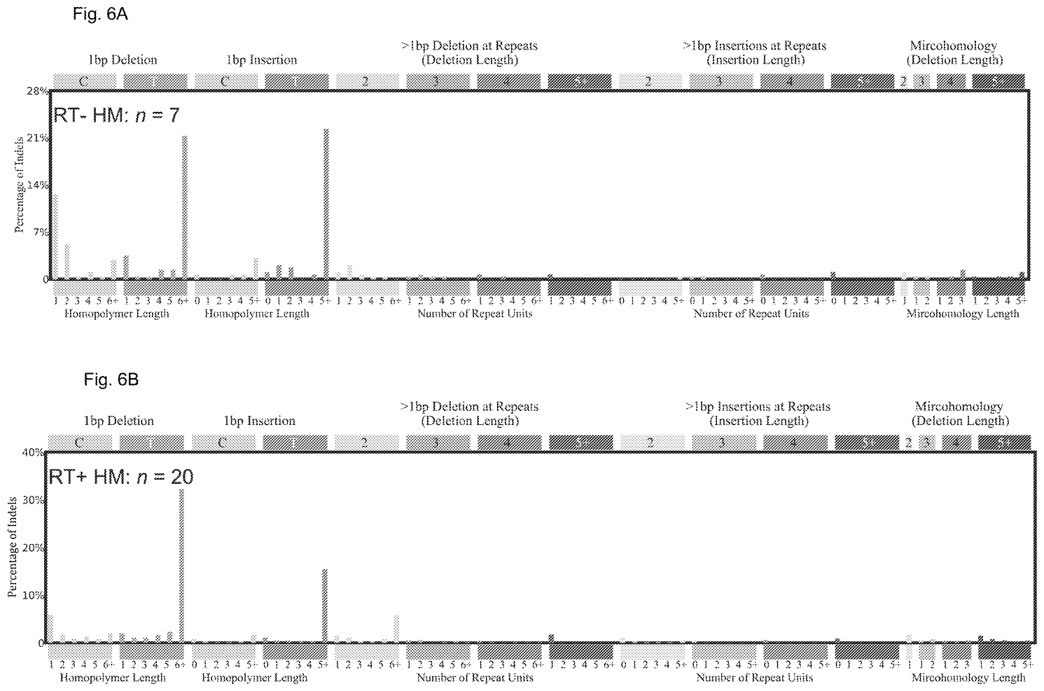

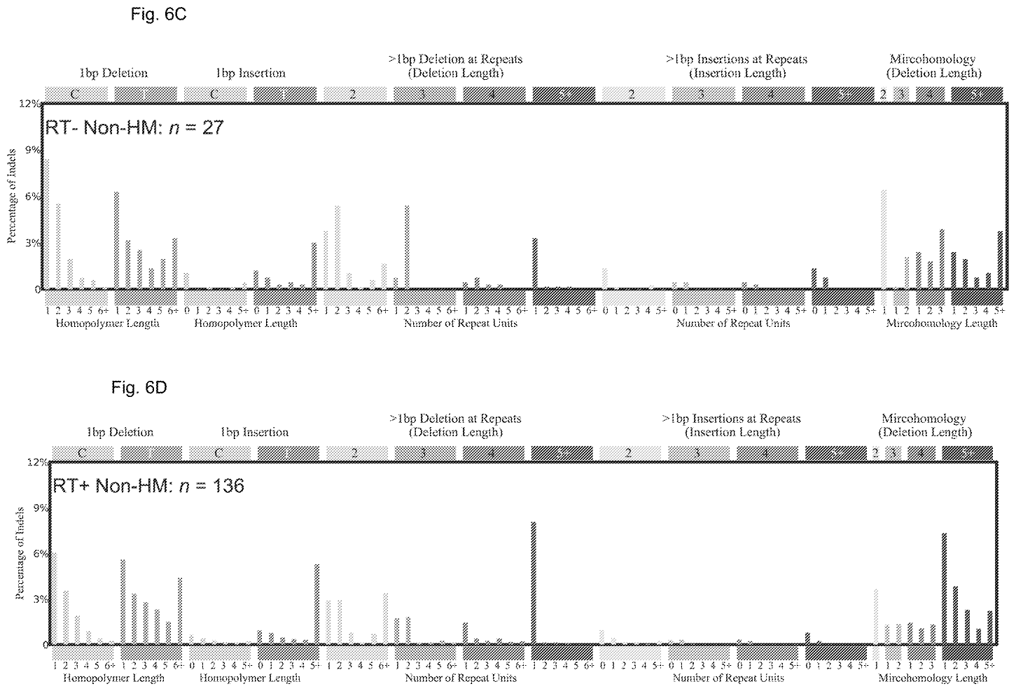

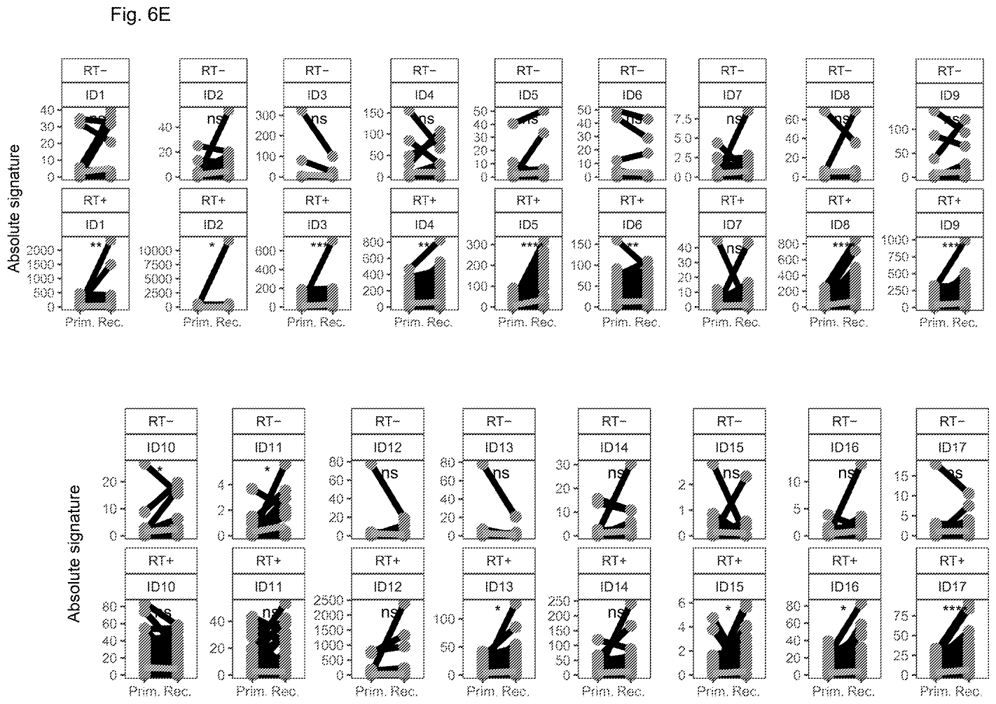

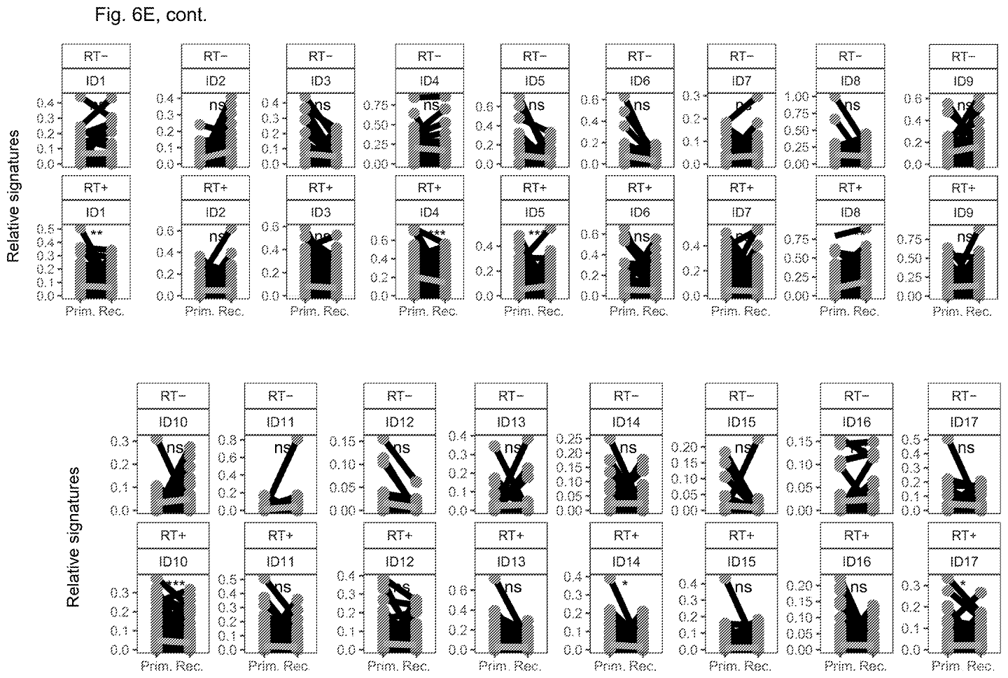

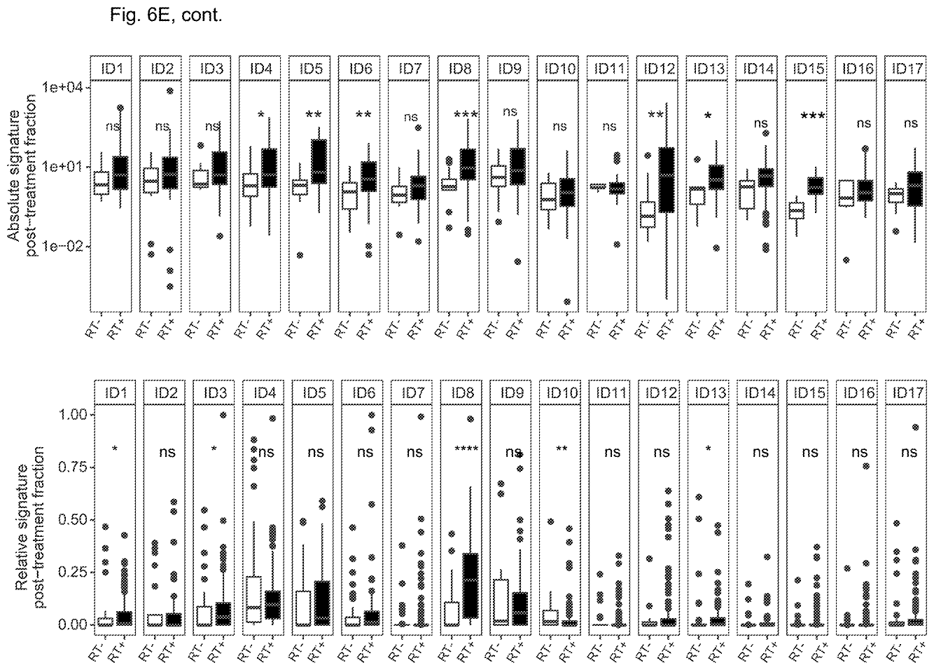

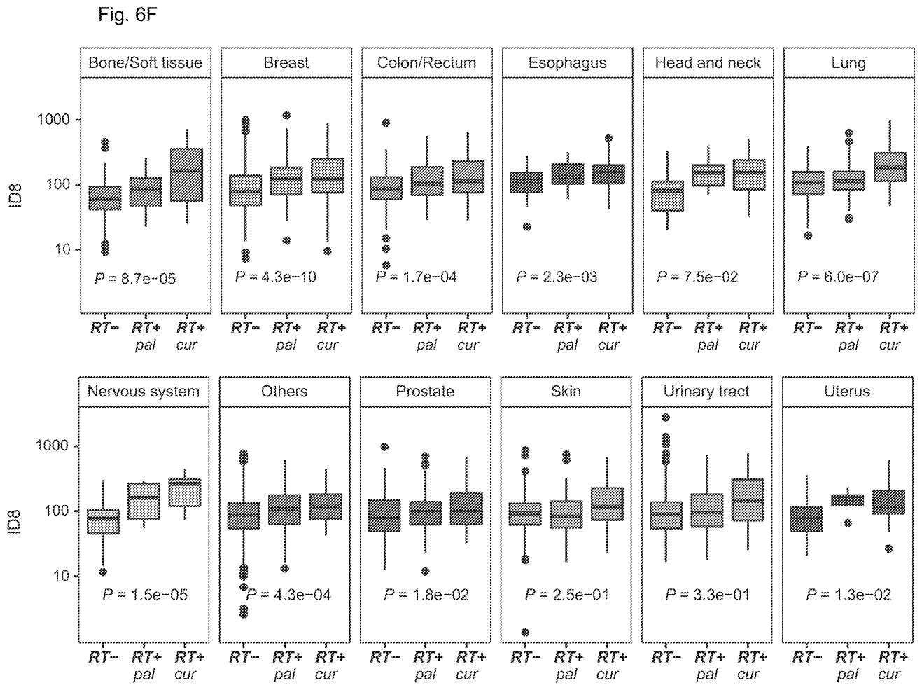

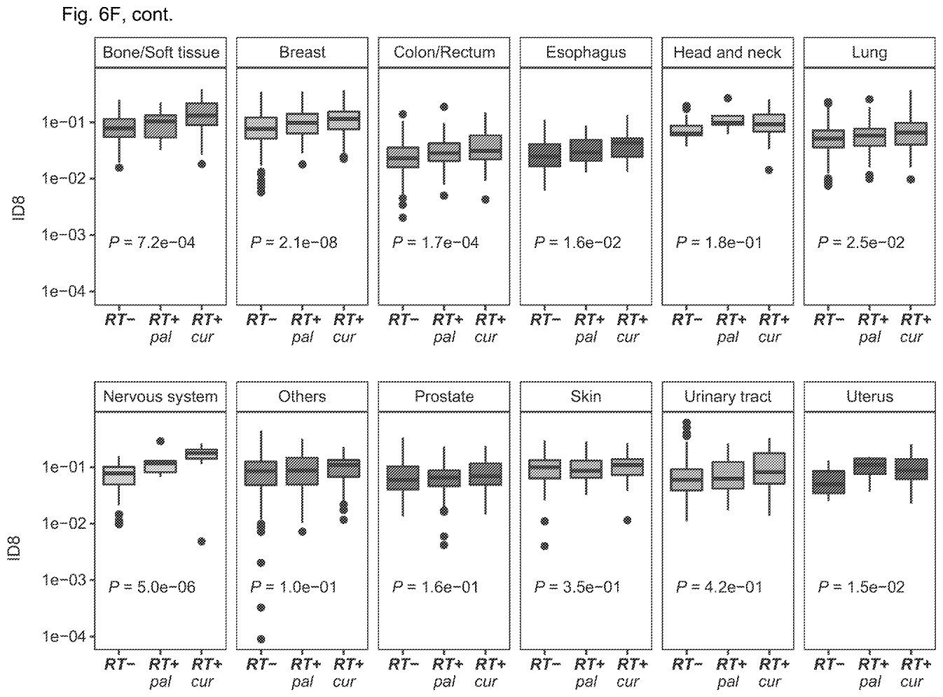

Mutational Signatures SigProfiler was used to extract and plot mutational signatures of single base substitutions (SBS), double base substitutions (DBS), and indels (ID) as previously described [Alexandrov, L. B. et al., Nature 578, 94-101 (2020)]. Absolute and relative contributions of signatures were determined using modified functions from the MutationalPatterns R package [Blokzijl, F., et al., Genome Med. 10, 33-33 (2018)]. Briefly, the mutational profile matrix generated with SigProfiler was fitted to the catalog of previously identified COSMIC mutational signatures (v3, May 2019) by solving the non-negative least squares problem. The single base substitution signatures SBS31 and SBS35 were previously linked to platinum therapy [Pich, O. et al., Nat. Genet. 51, 1732-1740 (2019); Alexandrov, L. B. et al., Nature 578, 94-101 (2020)]. Analysis of the HMF cohort using the extracted signatures confirmed these previously established associations, supported the identified signatures. SigProfilerPlotting [Bergstrom, E. N. et al., BMC Genomics 20, 685 (2019)] was used to visualize the distribution of indel characteristics (FIG. 6A-D).

### Results

Cancer cells accumulate somatic mutations that are caused by intrinsic and/or extrinsic mechanisms. The different mutational processes can leave distinct genomic scars, termed mutational signatures.

To validate the underlying mutational processes of radiotherapy, pre- and post-treatment mutations in the GLASS dataset were compared to previously defined mutational signatures [Alexandrov, L. B. et al., Nature 578, 94-101 (2020)]. The comparison of signature contributions between post-treatment mutations in RT-treated and RT-naïve IDH mutant glioma samples revealed a strong enrichment of indel signature 8 (ID8, FIG. 5, FIG. 6D, RT+, mean contribution=0.22, vs. RT−, mean contribution, P=7.4e-05, Q=3.8e-03, Mann-Whitney U test and false discovery rate, respectively). Furthermore, in RT-treated patients but not RT-naïve patients comparing ID8 values before and after treatment resulted in significant increases in absolute (FIG. 6E, P=4.5e-07, Paired Wilcoxon rank-signed test) and relative (FIG. 6E, P=2.3e-03) ID8 contributions, respectively. Signature ID8 was composed of >5 bp deletions without microhomology and had previously been linked to DSB repair by c-NHEJ, providing further evidence that radiation-induced DSBs were primarily repaired via c-NHEJ [Alexandrov, L. B. et al., Nature 578, 94-101 (2020)]. Hypermutation in IDH mutant gliomas was associated with a significant enrichment of indel signature 2 (ID2, FIG. 5, FIG. 6A-B). ID2 comprised 1-bp deletions at homopolymers and had been reported previously to be elevated in DNA mismatch repair deficient cancers [Alexandrov, L. B. et al., Nature 578, 94-101 (2020)].

A previous analysis of mutational signatures in the HMF dataset found that of all indel signatures the strongest association with radiotherapy treatment was with COSMIC-ID6 (corresponding to SignatureAnalyzer-ID12) [Pich, O. et al., Nat. Genet. 51, 1732-1740 (2019)]. Consistent with findings in the GLASS cohort, it was observed that the strongest association was with ID8, and significant but substantially less pronounced for ID6 (FIG. 5). Both absolute and relative ID8 values were significantly higher in RT-treated samples when compared to RT-naïve samples, and a significant association was observed in nine of twelve tumor types (FIG. 6F). Importantly, the comparison of HRD- and HRD− samples showed a clear association of HR-deficiency with ID6. ID6 comprised >5 bp deletions with microhomology at breakpoints and had previously been reported to be elevated in HR-defect breast cancers [Davies, H. et al., Nat. Med. 23, 517-525 (2017)]. Additionally, the findings from the GLASS cohort and previous observations that MSI samples were enriched for indel signature 2 (ID2, FIG. 5) were validated.

Collectively, these results have important implications for differential mutational processes acting on the cancer genome. While MSI leads to an increased burden in small deletions due to hypermutability resulting from impaired DNA mismatch repair at microsatellites/homopolymers, the DSBs induced by RT and due to HRD are repaired via error prone DSB-repair mechanisms. These results clearly suggested two different mechanisms for the repair of DSBs: the a-EJ pathway that utilizes microhomologies at breakpoints in HR-deficient samples (signature ID6) and the c-NHEJ pathway that does not require microhomologies at breakpoints in RT-treated samples (signature ID8).

Next, identification of single base substitution (SBS) signature associations in both datasets was investigated. Consistent with previous reports, significant enrichment of SBS11 was found in hypermutant IDH mutant glioma samples [Barthel, F. P. et al., Nature 576, 112-120 (2019); Touat, M. et al., (2020) Nature 580(7804), 517-523] and enrichment of signatures SBS44, SBS26, SBS21, SBS20, and SBS15 was found in MSI samples [Alexandrov, L. B. et al., Nature 578, 94-101 (2020)]. In HRD cases enrichment of SBS3 and SBS8 was observed, which was previously described [Alexandrov, L. B. et al., Nature 578, 94-101 (2020); Davies, H. et al., Nat. Med. 23, 517-525 (2017); Nik-Zainal, S. et al., Nature 534, 47-54 (2016)] along with a so far unreported enrichment of SBS39 (FIG. 5A-B). In addition to these confirmatory results, several previously unreported associations were found suggesting the involvement of APOBEC in RT-associated DSB-repair. In the GLASS cohort, RT-treatment was significantly associated with SBS13 and in the HMF cohort with SBS2 and SBS13. SBS2 and SBS13 are APOBEC signatures [Alexandrov, L. B. et al. Nature 578, 94-101 (2020) and Roberts, S. A. et al. Nat Genet 45, 970-6 (2013)]. APOBEC cytosine deaminases are involved in retrovirus and retrotransposon restriction, and the enrichment of APOBEC signatures in RT-treated samples in both datasets implicated APOBEC-mediated mutagenesis in association with RT-associated DSB-repair [Lei, L. et al. Nat Struct Mol Biol 25, 45-52 (2018); Nowarski, R. & Kotler, M. Cancer Res 73, 3494-8 (2013), and Nowarski, R. et al. Blood 120, 366-75 (2012)].

These results support the hypothesis that mutational signatures are shaped by cycles of DNA damage and DNA repair [Volkova, N. V. et al. Nat Commun 11, 2169 (2020]). While extrinsic radiotherapy causes DNA DSBs, the intrinsic erroneous repair of the damage via c-NHEJ leads to specific small deletions (ID8), and APOBEC cytidine deaminases may be activated during the repair process leading to specific SNVs (SBS2/SBS13).

### Example 4

Radiotherapy Treatment is Associated with Aneuploidy and Larger Deletions

### Materials and Methods

Materials and methods were as described in Examples 1-3 above herein, and as below herein, as appropriate.

### Structural Variants

For the GLASS dataset split reads and discordant read pairs were extracted from all tumor and normal BAM files using samtools 1.7 [Li, H. et al., Bioinformatics 25, 2078-9 (2009)]. The lumpyexpress tool (from LUMPY 0.2.13) was used to call structural variants providing the data associated with the set of normal and tumor samples belonging to one patient [Layer, R. M. et al., Genome Biol 15, R84 (2014)]. CNV predictions inferred from read-depth using CNVnator 0.3.3 were additionally provided to further support identified variants [Abyzov, A. et al., Genome Res. 21, 974-84 (2011)]. The resulting call set was post-processed using SVtyper 0.6.0 to genotype structural variants for each individual sample belonging to a patient [Chiang, C. et al., Nat. Methods 12, 966-8 (2015)]. Finally, GATK VariantFiltration was used to filter all variants with less than four reads of support and those with quality scores less than ten [Van der Auwera, G. A. et al., Curr. Protoc. Bioinformatics 11.10.1-11.10.33 (2013)]. Variants that showed any support in non-tumor samples were additionally removed. Variants were quantified per sample and further stratified according to type (translocation, duplication, deletion, and inversion). The change in frequencies for each patient was computed by dividing the rate at recurrence by the rate at primary. Only variants spanning at least 20 bp were considered.

### Aneuploidy Calculation

Arm-level aneuploidy data from the GLASS dataset was obtained from a previous publication and copy number segmentation files from HMF were processed into arm-level copy number calls as previously described [Barthel, F. P. et al., Nature 576, 112-120 (2019)]. Chromosomes demonstrating euploidy in both arms were considered euploid. Chromosomes with equidirectional aneuploidy in both arms or aneuploidy in a single arm and indeterminate ploidy in the other arm were considered “simple aneuploid”. Chromosomes with aneuploidy in one arm and incongruent ploidy in the other arm were considered “complex aneuploid”. Aneuploidy events were quantified for each tumor sample.

### Results

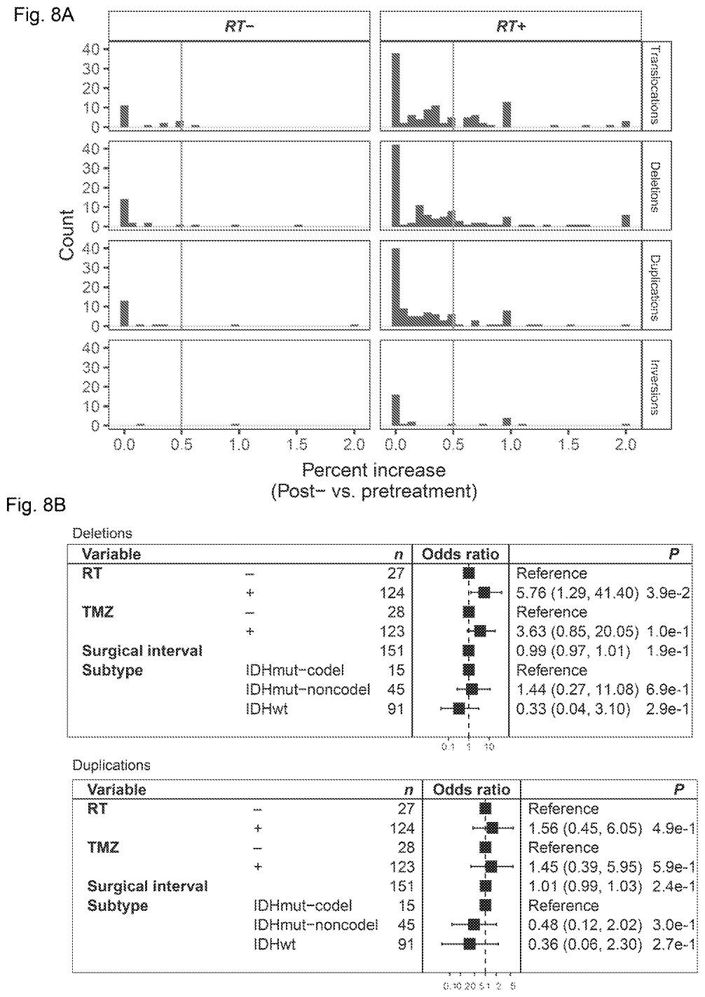

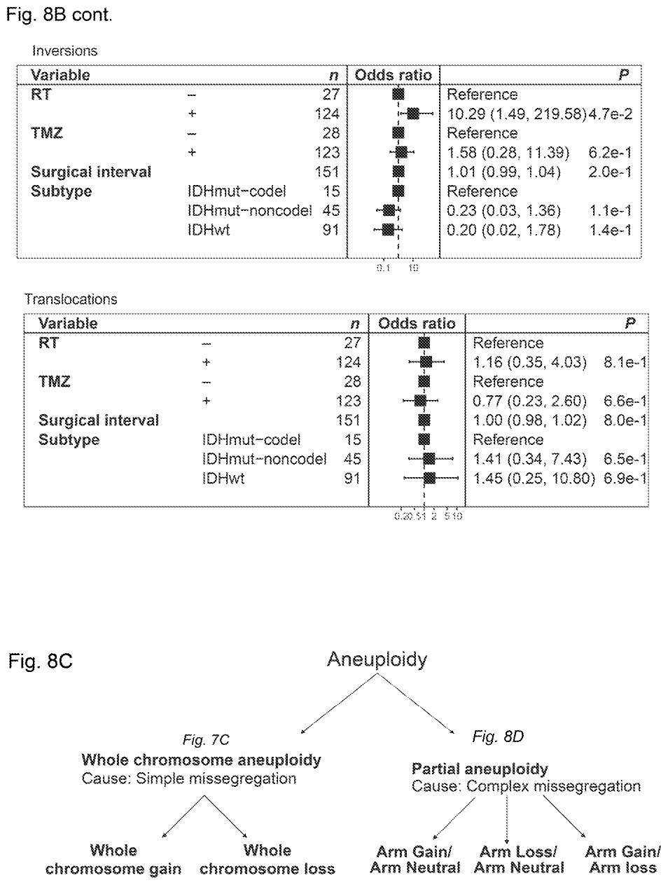

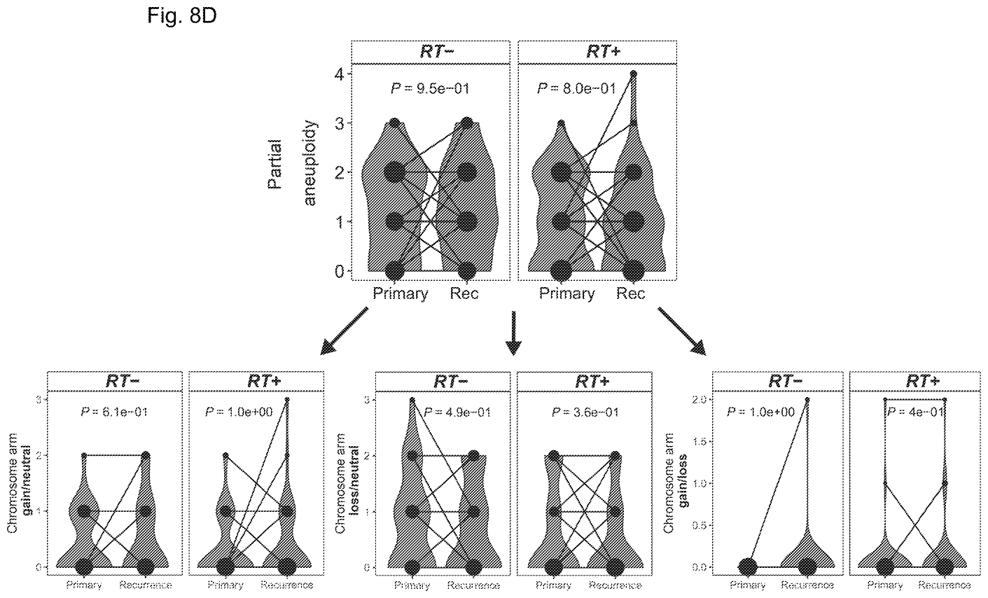

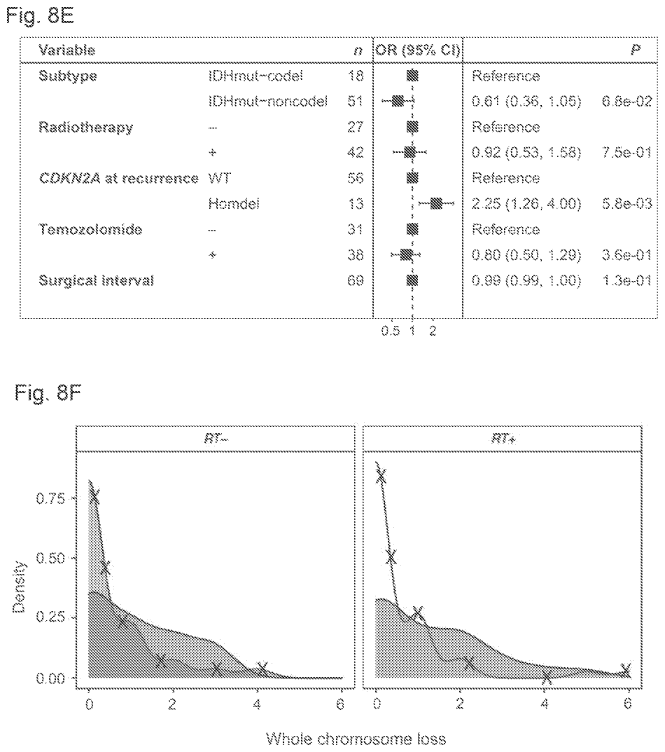

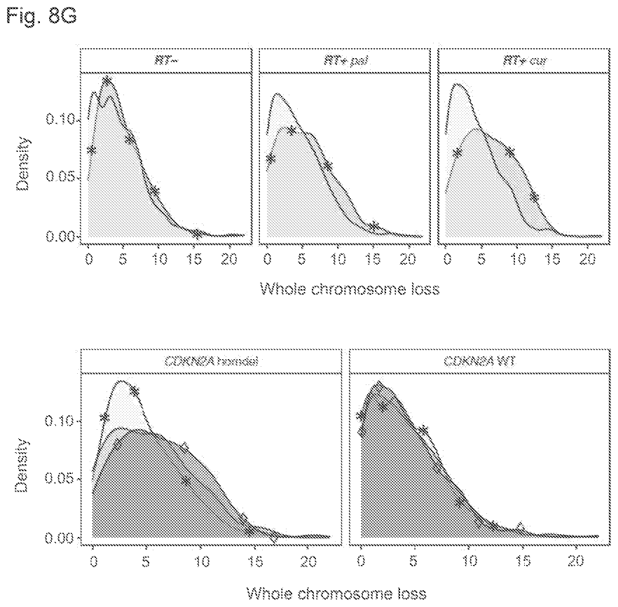

Having established an association with radiotherapy and small deletions as well as specific mutational signatures, it was reasoned that RT-induced DSBs may also result in other types of genomic damage. Large structural variants, including large deletions, duplications, inversions, and translocations, were detected in the longitudinal GLASS cohort, and it was observed that a statistically significant number of RT-treated patients demonstrated an increase of large deletions (length >20 bp to chromosome arm length) post-therapy that was not seen in RT-naïve patients (FIG. 7A, P=3.2 e-02, Fisher's exact test). Interestingly, a similar statistically significant increase in inversions was observed (FIG. 7A, P=2.1e-02), and no differences were observed for translocations (FIG. 7A, P=1) and duplications (FIG. 7A, P=7e-01). These associations remained significant after accounting for potentially confounding factors such as TMZ treatment and molecular subtype (FIG. 8B). While radiation-associated secondary malignancies have been reported to contain increased rates of inversions [Behjati, S. et al., Nat. Commun. 7, 12605 (2016)], a concomitant increase in large deletions in association with RT has not been previously observed.

The next evaluation was whether any specific deletions were associated with radiotherapy treatment. Comparing alteration frequencies before and after RT in the GLASS cohort identified a significant link between radiotherapy and gain of CDKN2A homozygous deletions among IDH-mutant gliomas where (′DKN2A loss at diagnosis is rare [Ceccarelli et al., 2016, Cell 164, 550-563] (FIG. 5B). The IDH-wild type gliomas subgroup in the GLASS cohort lacked the number of RT− cases to perform the same analysis. CDKN2A homozygous deletions occurred exclusively in RT-treated recurrences (FIG. 7B) and occurred significantly more frequently than in pre-treatment samples (FIG. 7B, 29% vs. 2%, P=1.9e-05, Fisher's exact test), nominating acquired (′DKN2A homozygous loss as a potential novel biomarker for RT-resistance among IDH-mutant gliomas, but not IDH-wild type gliomas where CDKN2A homozygous deletion at diagnosis is common.

Ionizing radiation can promote mitotic chromosome segregation errors causing aneuploidy [Adewoye, A. B., et al., Nat. Comm. 6, 6684-6684 (2015); Rose Li, Y., et al., Nat. Comm. 11, 394-394 (2020); Bakhoum, S. F., et al., Nat. Comm. 6, 5990-5990 (2015); Touil, N. et al., Mutagenesis 15, 1-7 (2000)]. Specifically, ionizing radiation can induce non-disjunction events during mitosis, leading to an imbalanced chromosomal copy number between two daughter cells [Behjati, S. et al., Nat. Commun. 7, 12605 (2016)]. The association of aneuploidy with radiation therapy was investigated, separating aneuploidy events into gains or losses of entire chromosomes, likely the result of segregation errors, and partial gains or losses, requiring additional DSBs (FIG. 8C). In an analysis of the IDH-mutant GLASS cohort, a significant association was observed between RT and chromosome losses, whereas no association was observed for simple gains or complex events (FIG. 7C, FIG. 8D). However, after adjusting for covariates in a multivariable Poisson regression model used to model integer counts of aneuploidy events, the effect of radiotherapy on chromosome losses in the GLASS cohort was no longer statistically significant. The analysis highlighted a significant association between chromosome losses and CDKN2A deletions (FIG. 8E), implicating that the increase in chromosome loss frequency following RT is specific to RT-associated acquired CDKN2A deletions. Using the Hartwig metastatic tumor cohort, an association between CDKN2A homozygous deletions and simple chromosome losses was demonstrated (FIG. 7D, FIG. 8F). Using Poisson regression to model associations of chromosome loss frequency showed that both curative RT-treatment and CDKN2A homozygous deletions were independently associated with increased number of chromosomal losses in the HMF dataset (FIG. 7D, FIG. 8F). However, testing for interactions between CDKN2A deletions and RT-treatment indicated a trend towards interaction between palliative/curative radiotherapy and CDKN2A deletions (Table 1, P=9.75e-02 and P=4.92e-02, respectively, t-test). As shown in Table 1, CDKN2A homdel and curative RT were independently associated with higher whole chromosome losses. These results suggest that chromosome segregation may not directly be associate with radiotherapy but through interactions with CDKN2A deletions. Thus, whereas radiotherapy may promote mis-segregation alone, when combined with CDKN2A homozygous deletions, this further exacerbates segregation errors and may generate additional aneuploidy.

TABLE 1
Multivariable Poisson regression model for whole chromosome losses
in metastatic cohort including tumor type, RT, CDKN2A status,
and an interaction term between RT and CDKN2A as variables.

| | | | Odds Ratio | |
|---|---|---|---|---|
| Variable | Levels | n | (95% CI) | P-value |
| Tumor type | Bone/Soft | 179 | (ref) | |
| | tissue | | | |
| | Breast | 716 | 1.10 (1.01-1.20) | 2.50E−02 |
| | Colon/Rectum | 445 | 1.42 (1.30-1.54) | 1.11E−15 |
| | Esophagus | 128 | 1.89 (1.71-2.08) | <2E−16 |
| | Head and Neck | 53 | 1.05 (0.90-1.22) | 5.12E−01 |
| | Lung | 355 | 1.73 (1.59-1.88) | <2E−16 |
| | Nervous | 74 | 0.51 (0.43-0.60) | 2.45E−14 |
| | System | | | |
| | Others | 810 | 1.16 (1.07-1.26) | 2.90E−04 |
| | Prostate | 392 | 0.53 (0.48-0.59) | <2E−16 |
| | Skin | 324 | 0.86 (0.78-0.94) | 1.50E−03 |
| | Urinary Tract | 163 | 1.05 (0.94-1.17) | 3.87E−01 |
| | Uterus | 54 | 1.03 (0.88-1.20) | 7.37E−01 |
| Radiotherapy | RT− | 2265 | (ref) | |
| | RT+ pal | 689 | 1.04 (0.99-1.08) | 1.30E−01 |
| | RT+ cur | 739 | 1.05 (1.01-1.10) | 2.40E−02 |
| CDKN2A status | WT | 3065 | (ref) | |
| | Homdel | 628 | 1.20 (1.14-1.26) | 1.79E−12 |
| | RT+ pal: | | | |
| Interaction terms | CDKN2A | | | 9.75E−02 |
| | homdel | | | |
| | RT+ cur: | | | |
| | CDKN2A | | | 6.92E−02 |
| | homdel | | | |

### Example 5

### RT-Driven Genomic Changes Result in Poor Survival

### Materials and Methods

Materials and methods were as described in Examples 1-4 above herein, as appropriate.

### Results

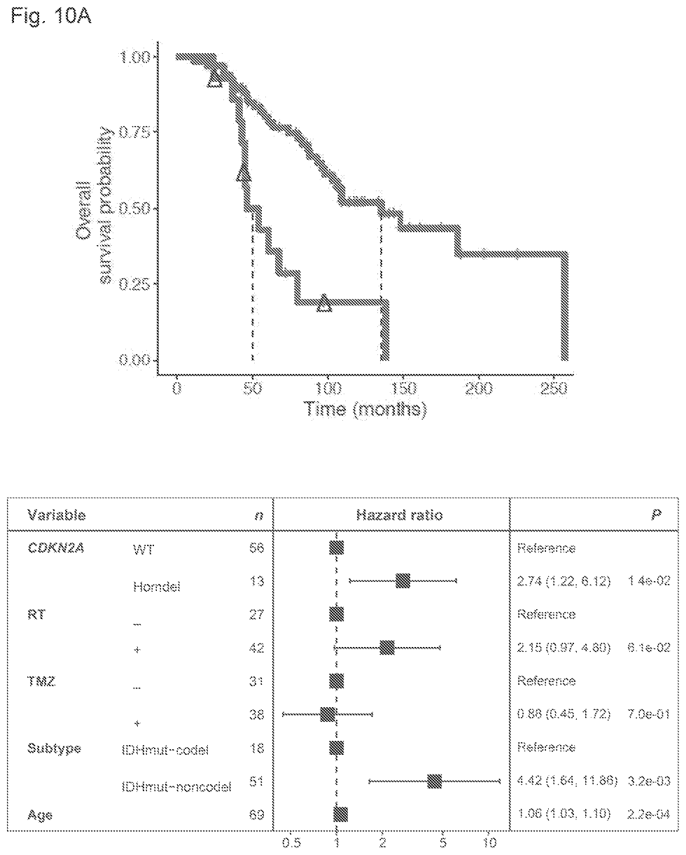

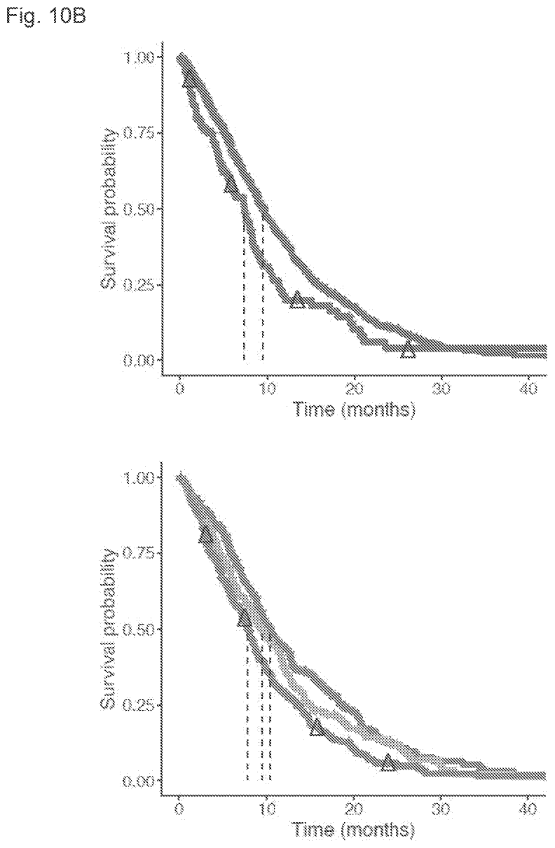

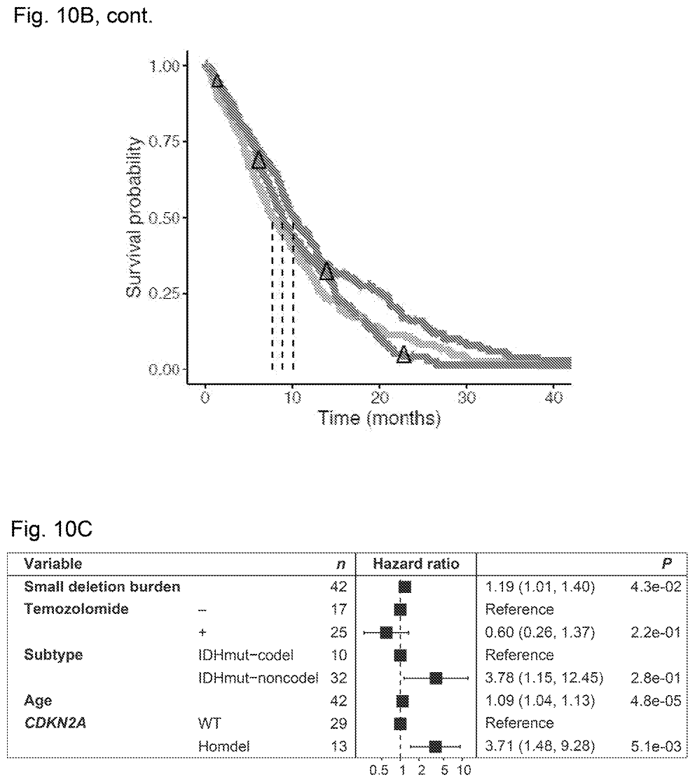

The final goal was to ascertain whether the genomic effects of radiotherapy were relevant to patient outcome. Survival analysis confirmed that CDKN2A homozygous deletion at recurrence was significantly associated with worse overall survival in IDH-mutant glioma samples (FIG. 10A, upper, P<1e-04, log-rank test), independent of age, treatment, or subtype (FIG. 10A, lower, P=1.4e-02, Wald test) [Barthel, F. P. et al., Nature 576, 112-120 (2019)]. To test for a survival association of CDKN2A deletions amongst RT-treated patients in the HMF dataset, 958 samples that received RT and had sufficient survival information available were selected from 11 tumor types (FIG. 2E). Although CDKN2A homozygous deletions are less common outside glioma and HMF data lacks longitudinal samples to limit the analysis to cases with acquired CDKN2A deletion, patients whose tumors harbored a CDKN2A homozygous deletions showed worse outcomes compared to patients with CDKN2A wild-type tumors (FIG. 10B, first panel). Stratification of the cohort into tertiles based on genome-wide aneuploidy frequency demonstrated that low aneuploidy was linked to favorable outcomes and high aneuploidy was linked to poor outcomes (FIG. 10B, second panel). These results indicated that acquired CDKN2A homozygous deletion was a biomarker of RT resistance after recurrence and supported the clinical reassessment of CDKN2A status at recurrence for optimizing treatment strategies, particularly not only as single time point as previously reported [Appay, R., et al., Neuro-oncology 21, 1519-1528 (2019); Shirahata, M., et al., Acta Neuropathologica 136, 153-166 (2018); van Thuijl, H. F., et al., Genome Biology 15, 471-471 (2014)]-but also longitudinal prognostic markers in IDH-mut glioma.

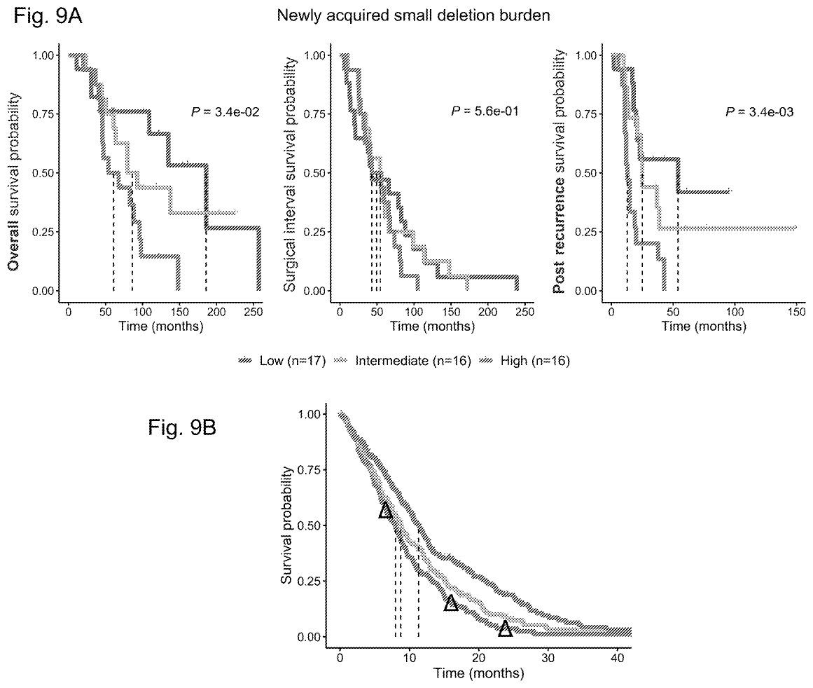

Independent of the poor prognostic implications of specific. RT-induced changes such as CDKN2A deletions, it was found that GLASS patients with tumors carrying a high small deletion frequency at recurrence (top tertile) had significantly shorter overall survival (FIG. 9A, P=3.4e-02, log-rank test). The association remained significant when accounting for the small deletion burden as a continuous variable and possible confounding variables, indicating a robust correlation (FIG. 10C, HR=1.19 [95% CI: 1.01-1.14]; P=4.3e-02, Wald test). Multivariable modeling using a limited subset of patients with detailed dosage information in the GLASS cohort (n=21) further indicated that the association with the small deletion burden and survival was independent of dose, P=2e-02). Separating the overall survival time into surgical interval and post-recurrence survival indicated that the association of high newly acquired small deletion burden with worse survival was limited to post-recurrence survival (FIG. 9A, P=3.4e-03, log-rank test). Surgical interval times did not differ significantly between the three tertiles (FIG. 9A, P=5.6e-01), suggesting that glioma patients may initially benefit equally from RT, but after certain exposure to RT and acquisition of the deletion signature, patients may lose sensitivity to further radiotherapy.

The association between radiotherapy and patient outcome in the HMF metastatic tumor datasets was then determined using the 958 RT-treated patients described above herein (FIG. 2G). Using this cohort, patients harboring a high small deletion burden (top tertile) were found to have significantly shorter survival than other RT-treated cases (FIG. 9B, P<4e-04, log-rank test). Similarly, HMF patients could be stratified into tertiles according to ID8 burden to show that an intermediate or high ID8 burden was associated with poor survival and a low ID8 burden was associated with improved outcomes (FIG. 10B, third graph). The effect of RT-associated small deletion burden on the survival of the patients was further independent of mutations in DDR genes. This validation of a worse outcome association in a single-time point analysis suggested that the presence of a higher number of RT-associated small deletions implicated a tumor that had responded to therapy, but which may have lost some or all of the treatment sensitivity. Taken together, these results support a finding that a higher deletion burden reflects a scenario that is favorable to a tumor, characterized by proficient DNA repair resulting in less tumor cell killing and decreased treatment efficacy, and that a higher deletion burden as a result of radiation therapy reflects a more aggressive tumor with increased levels of resistance to follow up treatments.

### Discussion

The studies described above herein in Examples 1-5 comprehensively evaluated the effects of ionizing radiation on the cancer genome using a cohort of pre- and post-treatment glioma samples and a cohort of metastatic tumor specimens. A unique signature of RT-associated deletions was identified. It was found that CDKN2A homozygous deletions were acquired in RT-treated IDH-mutant gliomas but not in untreated recurrent IDH-mutant gliomas, supporting a conclusion that radiotherapy-induced DNA damage promoted the acquisition of this poor prognostic marker. Further, it was found that a higher load of RT-induced deletions associated with worse patient outcomes, supporting a conclusion that the increased deletion burden reflected the repair of RT-induced DNA damage.

The genomic impact of therapeutic radiation has not been previously comprehensively shown, implicating a knowledge gap. Radiotherapy is used in the clinical management in over 50% of cancer patients [Barton, M. B. et al. Radiother. Oncol. 112, 140-4 (2014); Tyldesley, S. et al., Int. J. Radiat. Oncol. Biol. Phys. 79, 1507-15 (2011)] and effectively the most widely used regiment for cancer treatment. Prior studies on radiation induced tumors have shown a wide range of genomic effects and have suggested the involvement of various DNA double strand break repair mechanisms [Rose Li, Y. et al., Nat. Commun. 11, 394 (2020); Behjati, S. et al., Nat. Commun. 7, 12605 (2016); Davidson, P. R. et al., Sci. Rep. 7, 7645 (2017); Lopez, G. Y. et al., Acta Neuropathol. 137, 139-150 (2019); Phi, J. H. et al., Acta Neuropathol. 135, 939-953 (2018)]. The findings herein that RT was associated with a significantly higher burden of small deletions harboring specific genomic signatures, large deletions, large inversion and whole chromosome loss-driven aneuploidy extends the knowledge base and provides direction for development of effective radiosensitizers.

This work supports a conclusion that these events are a consequence of RT-induced mutagenesis/repair cycles. While the nearly exclusive association between acquired CDKN2A deletions and RT-treatment supports a conclusion that these events are RT-induced. The significant expansion of clones harboring RT-induced genomic events depends on clonal selection or drift [Reiter, J. G. et al., Nat. Genet. 52, 692-700 (2020)]. Therefore, the increased small deletion burden in combination with poor outcomes appeared to reflect the emergence of more competitive clones under RT-induced stress, innately active repair processes ensuring tumor maintenance, or a combination of these two. Thus, additional rounds of RT in patients with recurrent or metastatic tumors containing a significant increase in small deletion burden is unlikely to further extend progression-free survival. The assessment of small deletions may be used as a means with which to readily detect increased small deletion burden may help reduce costs of treatment and avoid RT-associated patient comorbidities and side-effects.

The studies describe herein resulted in identification of enrichment of APOBEC associated mutational signatures and thus supports a role for inhibitors of this class of cytosine deaminases as radiosensitizers. Compounds that inhibit DNA repair can be used to improve the response of cancer cells to radiotherapy. The instant studies indicate the importance of effective DNA repair in therapy resistance.

### Example 6

Detecting a ‘Small Deletion Phenotype’ from Circulating Tumor DNA and Determining Whether Additional Radiation Treatment could be Effective

Circulating tumor DNA (ctDNA) is isolated from blood or cerebrospinal fluid using commercially available methods such as from Qiagen (Qiagen, Germantown, MD), and may be used to identify tumor-type-specific signatures [Nassiri et al., Nat. Med. 26, 1044-1047 (2020)]. The burden of small deletions, a normalized approximation of the total number of small deletions across the genome, is compared to the burden of small deletions detected in the genome of a tumor specimen. When the positive difference between ctDNA-derived and tumor specimen-derived small deletion burden exceeds a certain threshold, which is either an absolute (+0.5 small deletions/Mb) or a relative increase (50% additional new small deletions), it may be decided that additional treatment regimens based on ionizing radiation will not be effective. Thus, information obtained from assessing the burden of small deletions in a subject is used to select a treatment regimen for the subject, and the subject is administered the selected treatment regimen.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method of radio-sensitizing a cell, comprising: contacting the cell with an exogenous cytidine deaminase inhibitor compound.

2. The method of claim 1, wherein the exogenous cytidine deaminase inhibitor compound is capable of inhibiting an apolipoprotein B mRNA editing enzyme, catalytic polypeptide-like (APOBEC) enzyme.

3. The method of claim 2, wherein the APOBEC enzyme is one or more of: an APOBEC1 enzyme, an APOBEC3A enzyme, an APOBEC3B enzyme, an APOBEC3C enzyme, an APOBEC3D enzyme, an APOBEC3E enzyme, an APOBEC3F enzyme, an APOBEC3G enzyme, an APOBEC3H enzyme, and an activation-induced cytidine deaminase (AID) enzyme.

4. The method of claim 1, wherein the cell is a cancer cell.

5. The method of claim 1, further comprising contacting the cell with radiation.

6. The method of claim 1, further comprising contacting the cell with two or more exogenous cytidine deaminase inhibitor compounds.

7. The method of claim 1, wherein the cell is in a subject.

8. The method of claim 7, wherein the contacting comprises administering the exogenous cytidine deaminase inhibitor compound to the subject.

9. The method of claim 7, wherein the subject has a cancer.

10. The method of claim 9, wherein the cancer is a cancer with elevated APOBEC activity.

11. The method of claim 9 or 10, wherein the cancer is a bone cancer, a soft tissue cancer, a colon cancer, a rectal cancer, an esophageal cancer, a lung cancer, a central nervous system (CNS) cancer, or uterine cancer.

12. The method of claim 9, wherein the cancer is breast cancer.

13. The method of claim 8, further comprising administering a radiotherapy to the subject.

14. The method of claim 13, wherein the radiotherapy comprises external beam radiation.

15. The method of claim 13, wherein the radiotherapy comprises brachytherapy.

16. The method of claim 7, wherein the subject is a vertebrate, optionally a mammal.

17. The method of claim 7, wherein the subject is a human.

18. The method of any one of claims 1-17, wherein the exogenous cytidine deaminase inhibitor compound is in a pharmaceutical composition and optionally the pharmaceutical composition further comprises a pharmaceutically acceptable carrier.

19. A method of enhancing efficacy of a radiotherapy administration in a subject, comprising:
administering to a subject in need of such treatment, an effective amount of an exogenous cytidine deaminase inhibitor compound, wherein
(i) the subject is administered a radiotherapy;
(ii) the exogenous cytidine deaminase inhibitor compound is administered in a therapeutic regimen; and
(iii) the administered exogenous cytidine deaminase inhibitor compound enhances the efficacy of the administered radiotherapy in the subject.

20. The method of claim 19, wherein the exogenous cytidine deaminase inhibitor compound is capable of inhibiting an apolipoprotein B mRNA editing enzyme, catalytic polypeptide-like (APOBEC) enzyme.

21. The method of claim 20, wherein the APOBEC enzyme is one or more of: an APOBEC1 enzyme, an APOBEC3A enzyme, an APOBEC3B enzyme, an APOBEC3C enzyme, an APOBEC3D enzyme, an APOBEC3E enzyme, an APOBEC3F enzyme, an APOBEC3G enzyme, an APOBEC3H enzyme, and an activation-induced cytidine deaminase (AID) enzyme.

22. The method of claim 19, wherein the therapeutic regimen comprises administering the exogenous cytidine deaminase inhibitor compound prior to or concurrent with the administered radiotherapy.

23. The method of claim 19, wherein the therapeutic regimen comprises administering the exogenous cytidine deaminase inhibitor compound to the subject prior to and concurrent with the administered radiotherapy.

24. The method of claim 19, wherein the subject is a vertebrate, optionally a mammal.

25. The method of claim 19, wherein the subject is a human.

26. The method of claim 19, wherein the enhancing increases a level of cell death in the subject compared to a control level of cell death.

27. The method of claim 26, wherein the cell death comprises cancer cell death.

28. The method of claim 26 or 27, wherein the control level of cell death is a level of cell death in a subject or plurality of subjects administered the radiotherapy and not administered the exogenous cytidine deaminase inhibitor compound therapeutic regimen.

29. The method of claim 19, wherein the radiotherapy comprises external beam radiation.

30. The method of claim 19, wherein the radiotherapy comprises brachytherapy.

31. The method of claim 19, wherein the subject has a cancer.

32. The method of claim 31, wherein the cancer is a cancer with elevated APOBEC activity.

33. The method of claim 31 or 32, wherein the cancer is a bone cancer, a soft tissue cancer, a colon cancer, a rectal cancer, an esophageal cancer, a lung cancer, a central nervous system (CNS) cancer, or uterine cancer.

34. The method of claim 31, wherein the cancer is breast cancer.

35. The method of claim 19, wherein the exogenous cytidine deaminase inhibitor compound is administered to the subject in a pharmaceutical composition, optionally wherein the pharmaceutical composition further comprises a pharmaceutically acceptable carrier.
